(19) United States
(12) Patent Application Publication
(10) Pub. No.: US 2024/0308946 A1
(43) Pub. Date: Sep. 19, 2024

(54) PRODUCTION OF LIGNIN DERIVED RENEWABLE LUBRICANTS

(71) Applicant: University of Delaware, Newark, DE (US)

(72) Inventors: Elvis Osamudiamhen Ebikade, Washington, DC (US); Sunitha Sadula, Wilmington, DE (US); Sibao Liu, Newark, DE (US); Dionisios G. Vlachos, Newark, DE (US)

(73) Assignee: University of Delaware, Newark, DE (US)

(21) Appl. No.: 18/681,356

(22) PCT Filed: Aug. 8, 2022
(86) PCT No.: PCT/US2022/039739

§ 371 Date: Feb. 5, 2024

Related U.S. Application Data
(60) Provisional application No. 63/230,157, filed on Aug. 6, 2021.

(51) Int. Cl. C07C 43/23; A61K 8/31; A61K 8/33; A61K 8/34; A61K 47/06; A61K 47/08; A61K 47/10; A61Q 19/00; C07C 13/28; C07C 41/30

(52) U.S. Cl. CPC C07C 43/23; A61K 8/31; A61K 8/33; A61K 8/347; A61K 47/06; A61K 47/08; A61K 47/10; A61Q 19/00; C07C 13/28; C07C 41/30; A61K 2800/10; A61K 2800/5922; C07C 2601/14

(57) ABSTRACT

Disclosed herein are bio-based compositions comprising branched aromatic compounds and/or branched cycloaliphatic compounds, and methods of their preparation from lignin-derived monomers, and their use as base oils in lubricant compositions, personal care compositions, and pharmaceutical compositions.

### CROSS REFERENCE TO RELATED APPLICATIONS

This application claims priority to U.S. Provisional Patent Application No. 63/230,157, filed Aug. 6, 2021, the entire disclosure of which is incorporated herein by reference for all purposes.

### STATEMENT REGARDING FEDERALLY SPONSORED RESEARCH

This invention was made with support from the government under DOE Grant No. DE-SC0001004 awarded by the Department of Energy. The government has certain rights in the inventions recited herein.

### FIELD OF THE INVENTION

The present invention relates to lubricant compositions and in particular to bio-based compounds for use in lubricant compositions and base oils for pharmaceutical and personal care product formulations, and methods of making such compounds.

### BACKGROUND OF THE INVENTION

Lubricants are widely used in industrial machinery, automobiles, aviation machinery, refrigeration compressors, agricultural equipment, marine vessels and many other applications and represent an over $126 billion global chemical enterprise. Base oils are key components (typically, 75-99 wt %) of commercial formulated lubricants and account for up to 75% of lubricant cost. Synthetic base oils of lower viscosity, such as poly-a-olefins (PAOs), are typically used in automobiles, whereas higher viscosity base oils, like alkylbenzenes, are used in cooling and refrigeration, delivering better thermal stability, higher lubricity, and lower hygroscopicity than mineral oils for higher compressor temperatures. PAO and alkylbenzene lubricant base oils obtained from petroleum feedstocks contribute significantly to greenhouse gas emissions. Renewable alternatives can mitigate CO₂ emissions. Synthesis of high-performance biolubricant base oils can be impactful.

Biolubricant base oils produced from furans-one of the most widely studied biomass-derived platform chemicals, possess excellent performance. Furans can be made from the carbohydrate fraction of biomass. Lignin is a naturally occurring crosslinked, functionalized biopolymer composed of three main aromatic monomers (p-coumaryl, coniferyl, and sinapyl alcohol) linked via various C—C and C—O bonds. It is the only natural source of aromatic monomers. Presently, pulping and biorefining industries produce approximately 70-100 Mt/y isolated lignin. Most isolated lignins have a dark color, strong odor, broad molecular weight distribution, and limited reactivity due to high fractions of recalcitrant C—C bonds, relegating to low-value applications (e.g., fillers for tires or burnt for energy). Despite advances in lignin depolymerization to high-yield aromatic monomers (hydroxyphenols, guaiacols, and syringols), harnessing these platform monomers beyond pharmaceuticals, polymers, and fine chemicals has received little attention. Hence, there is a need to synthesize bio-based lubricants and base oils from lignin-derived aromatic monomers with high selectivity and separation of the catalysts from the product.

### SUMMARY OF THE INVENTION

Accordingly, an object of the present invention is to provide novel strategies to produce lubricant base oils with tailored molecular architecture, tunable properties and content. In particular, the lubricant base oils disclosed herein will contain one or more branched aromatic compounds, branched cycloaliphatic compounds and/or branched hydrocarbon chains.

Disclosed herein are new strategies, such as shown in FIG. 1, to synthesize (1) branched benzene lubricant (BBL) base oils via the hydroxyalkylation/alkylation (HAA) of lignin-derived guaiacol and lauryl aldehyde and (2) branched cyclic lubricant (BCL) base oils via the hydrodeoxygenation (HDO) of the BBL products. While C—C coupling has been applied to upgrade furans into lubricants, application to lignin-derived monomers has rarely been exploited. Guaiacols are obtained either from woody or herbaceous biomass via reductive catalytic fractionation (RCF). Aldehydes of varying carbon length can be synthesized via dehydrogenation of biomass-derived alcohols or selective hydrogenation of fatty acids from natural oils or waste cooking oils (WCO). Optimum reaction conditions from guaiacol and lauryl aldehyde achieve a maximum guaiacol conversion of 90% with 76% BBL and 24% aldol condensation products over a P—SiO₂ catalyst. Subsequent HDO over an Ir—ReOx/SiO₂ catalyst produces a lubricant-ranged mixture of BCL (C24) at 82% yield and small fractions of dodecyl cyclohexane and C10 and C15 carbon alkanes. The lubricant properties determined such as the kinematic viscosity, viscosity index, and Noack volatility, show comparable performance to petroleum-derived PAO, alkylbenzene, and cyclic alkane base oils.

Disclosed herein is a bio-based composition comprising a branched aromatic compound having one of the following structures:

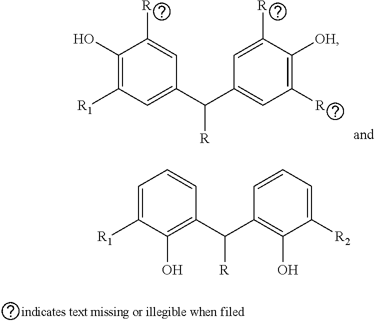

wherein R is an alkyl group having 1 to 20, or 3 to 19, or 5 to 17 carbon atoms,
wherein the alkyl group is a linear, a branched, or a cycloalkyl group, and
wherein R₁ and R₂ are independently selected from hydrogen and a methoxy group.

In an embodiment, the bio-based composition further comprises a bio-based aliphatic enal compound having the following structure:

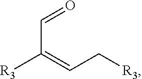

wherein each R₃ is independently selected from an alkyl group having 0 to 19, or 2 to 18, or 4 to 16 carbon atoms, and wherein the alkyl group is a linear, a branched, or a cycloalkyl group.

In another aspect, there is a bio-based composition comprising a branched cycloaliphatic compound having one of the following structures:

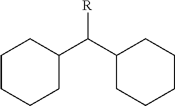

and isomers thereof, and

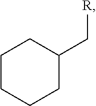

wherein R is an alkyl group having 1 to 20, or 3 to 19, or 5 to 17 carbon atoms, and wherein the alkyl group is a linear, a branched, or a cycloalkyl group.

In an embodiment of the bio-based composition comprising a branched cycloaliphatic compound, the composition may further comprise a branched aliphatic compound having the following formula:

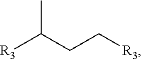

and isomers thereof,
wherein each R₃ is independently selected from an alkyl group having 0 to 19, or 2 to 18, or 4 to 16 carbon atoms, and wherein the alkyl group is a linear, a branched, or a cycloalkyl group.

The bio-based compositions as disclosed hereinabove may comprise at least one of compound having a bio-based content in the range of 20 to 100%, according to ASTM-D6866.

In another aspect, there is a bio-based lubricant composition comprising:
(i) a branched aromatic compound having one of the following structures:

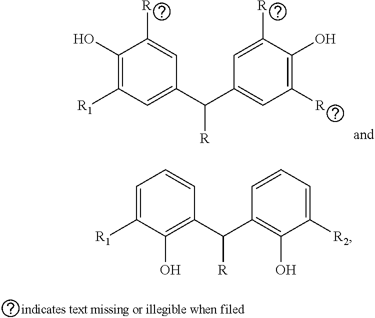

and optionally an aliphatic enal compound having the following structure:

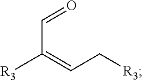

or
(ii) a branched cycloaliphatic compound having one of the following structures:

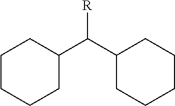

and isomers thereof, and

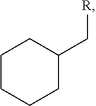

and optionally a branched aliphatic compound having the following structure:

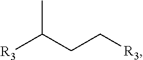

and isomers thereof,
wherein R is an alkyl group having 1 to 20, or 3-19, or 5-17 carbon atoms, wherein the alkyl group is a linear, a branched, or a cycloalkyl group, wherein R₁ and R₂ are independently selected from hydrogen and methoxy group, and wherein each R₃ is an alkyl group having 0 to 19, or 2 to 18, or 4 to 16 carbon atoms; and
(iii) an effective amount of one or more additives.

The one or more additives present in the bio-based lubricant as disclosed hereinabove, may be selected from the group consisting of antioxidants, stabilizers, detergents, dispersants, demulsifiers, antioxidants, anti-wear additives, pour point depressants, viscosity index modifiers, friction modifiers, anti-foam additives, defoaming agents, corrosion inhibitors, wetting agents, rust inhibitors, copper passivators, metal deactivators, extreme pressure additives, and combinations thereof.

In an embodiment, the bio-based lubricant composition of the present disclosure may further comprise one or more co-base oils selected from the group consisting of API Group I base oil, Group II base oil, Group III base oil, Group IV base oil, Group V base oil, gas-to-liquid (GTL) base oil, and combinations thereof.

In an embodiment, the bio-based lubricant composition has a kinematic viscosity at 100° C. in the range of 2 to 100 CSt, a kinematic viscosity at 40° C. in the range of 5 to 300 CSt, as measured by ASTM D445, and a viscosity index calculated from kinetic viscosity at 100° C. and 40° C., in the range of −40 to 200, as measured by ASTM D2270.

In yet another aspect, the bio-based lubricant composition as disclosed herein, is used in one or more of industrial machinery, automobiles, aviation machinery, refrigeration compressors, agricultural equipment, marine vessels, medical equipment, hydropower production machinery, and food processing equipment.

In a further aspect, there is a method of making a bio-based composition, such as the bio-based lubricant composition, as disclosed hereinabove, the method comprising carrying out hydroxyalkylation/alkylation of a lignin-derived monomer containing a phenolic hydroxyl group and an aldehyde (RCHO) in the presence of an acidic catalyst to form a bio-based branched aromatic compound having one of the following structures:

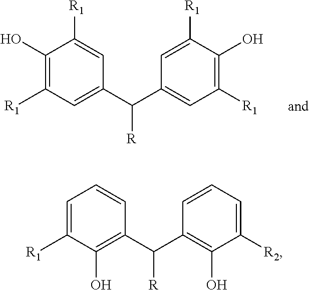

and optionally a branched aliphatic enal compound having the following structure:

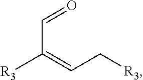

wherein R is an alkyl group having 1 to 20, or 3 to 19, or 5 to 17 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19, or 2 to 18, or 4 to 16 carbon atoms.

In an embodiment of the method of making a bio-based composition, the lignin-derived monomer comprises:
(i) an unsubstituted phenol or a substituted phenol, such as cresol (methyl phenol), ethylphenol, propylphenol,
(ii) a monomethoxyphenol, such as guaiacol (monomethoxy-substituted phenol), methylguaiacol, ethylguaiacol, propylguaiacol,
(iii) a dimethoxyphenol, such as a syringol (dimethoxy-substituted phenol), methyl syringol, or
(iv) combinations thereof.

The method of making a bio-based composition further comprises hydrodeoxygenating the bio-based aromatic compound in the presence of a hydrodeoxygenation catalyst to obtain a bio-based cycloaliphatic compound having the following formula:

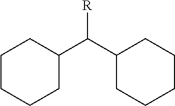

wherein R is an alkyl group having 1 to 20, or 3 to 19, or 5 to 17 carbon atoms, and wherein the alkyl group is a linear, a branched, or a cycloalkyl group.

In an embodiment of the method, the hydroxyalkylation/alkylation acidic catalyst is a homogeneous catalyst comprising sulfuric acid, methanesulfonic acid, acetic acid, triflic acid, or p-toluenesulfonic acid. In another embodiment, the hydroxyalkylation/alkylation acidic catalyst is a solid acid catalyst comprising perfluorinated sulfonic acid resins, sulfonic acid-functionalized cross-lined polystyrene resins, zeolites, or silica supported H₃PO₄.

In another embodiment of the method, the hydrodeoxygenation catalyst is a solid acid supported metal-based catalyst selected from Ni/ZSM-5, Pd/ZSM-5, Pd/BEA, or a physical mixture of a metal based catalyst with a solid acid, including Pd/C+ZSM-5, Pd/C+BEA, Pt/C+BEA, Pt-WOx/C and preferably a supported metal-metal oxide catalyst. Suitable supported metal-metal oxide catalyst comprises Ir—ReOₓ/SiO₂, Ir—MoOₓ/SiO₂ or ¹M²MO/SiO₂, wherein ¹M=Ir, Ru, Ni, Co, Pd, Pt, or Rh and ²M=Re, Mo, W, Nb, Mn, V, Ce, Cr, Zn, Co, Y, or Al.

In yet another aspect, the bio-based lubricant composition as disclosed herein, is used as a base oil in pharmaceutical and personal care products.

In an aspect, a personal care composition is provided, the personal care composition comprising:
a) a base oil comprising one or more of the following:
   (i) a branched aromatic compound having one of the following structures:

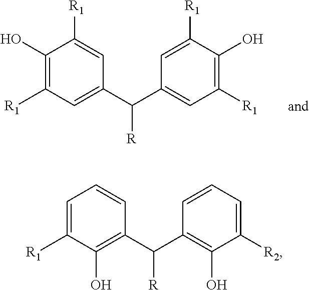

and optionally an aliphatic enal compound having the following structure:

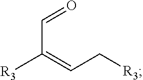

or
(ii) a branched cycloaliphatic compound having one of the following structures:

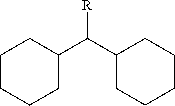

and isomers thereof, and

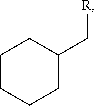

and optionally a branched aliphatic compound having the following structure:

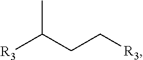

and isomers thereof,
wherein R is an alkyl group having 1 to 20, or 3 to 19, or 5 to 17 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19, or 2 to 18, or 4 to 16 carbon atoms;
b) an effective amount of one or more additives selected from the group consisting of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, and water; and
c) optionally, an active ingredient selected from the group consisting of antibiotic, antiseptic, antifungal, corticosteroid, and anti-acne agent.

In another aspect, a pharmaceutical composition is provided, the pharmaceutical composition comprising:
(a) a base oil comprising one or more of the following:
   (i) a branched aromatic compound having one of the following structures:

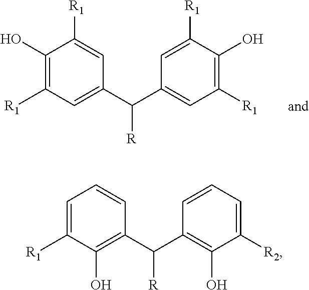

and optionally an aliphatic enal compound having the following structure:

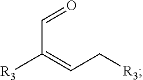

or
(ii) a branched cycloaliphatic compound having one of the following structures:

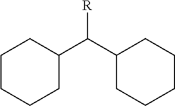

and isomers thereof, and

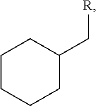

and optionally a branched aliphatic compound having the following structure:

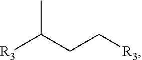

and isomers thereof,
wherein R is an alkyl group having 1 to 20, or 3 to 19, or 5 to 17 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19, or 2 to 18, or 4 to 16 carbon atoms;
(b) an effective amount of one or more pharmaceutically active ingredients; and
(c) optionally, one or more pharmaceutically acceptable excipients.

### BRIEF DESCRIPTION OF THE DRAWINGS

FIG. 1 shows a strategy to produce bio-based compositions such as branched benzene lubricant (BBL) base oils and branched cyclic lubricant (BCL) base oils via the hydroxyalkylation/alkylation (HAA) of lignin-derived monomers (e.g., hydroxyphenyls, guaiacols, and syringols) with an aldehyde and the hydrodeoxygenating (HDO) of the BBL product, respectively, in accordance with embodiments of the present invention.

FIG. 2 shows the BBL (2,2′-(1,1-dodecanediyl)-6,6′ -dimethoxydiphenol) yield, produced in a HAA reaction of guaiacol with lauryl aldehyde, over various acid catalysts (Reaction conditions: 10 mmol guaiacol, 5 mmol lauryl aldehyde, 8 hr, 150° C., 0.05 mmol H⁺).

FIG. 3 shows stoichiometric equations for the HAA (the two individual steps and the total) and the aldol condensation side reactions.

FIGS. 4A and 4B show the effect of temperature on the yield of the BBL (2,2′-(1,1-dodecanediyl)-6,6′-dimethoxydiphenol) and enal (13-decyl-12-ene-tetradecanal) products and conversion of the guaiacol and lauryl aldehyde reactants. (Reaction conditions for FIG. 4A: 10 mmol guaiacol, 5 mmol lauryl aldehyde, 12 hr, 150 mg P—SiO₂; Reaction conditions for FIG. 4B: 10 mmol guaiacol, 5 mmol lauryl aldehyde, 1 hr, 150 mg P—SiO₂).

FIGS. 5A and 5B show the effect of catalyst (P—SiO₂) amount on the yield of BBL and enal products and conversion of the guaiacol and lauryl aldehyde reactants. (Reaction conditions for FIG. 5A: 10 mmol guaiacol, 5 mmol lauryl aldehyde, 12 hr, 150° C. B) Reaction conditions for FIG. 5B: 10 mmol guaiacol, 5 mmol lauryl aldehyde, 1 hr, 150° C.).

FIG. 6 shows a gas chromatogram after heating lauryl aldehyde with and without acid catalyst. (Reaction conditions: 5 mmol lauryl aldehyde, 100 mg P—SiO₂, 150° C., 15 hr. Pictures inset from lauryl aldehyde condensation with and without acid catalyst).

FIG. 7 shows the effect of time on the yields of the BBL and enal products and conversion of the guaiacol and lauryl aldehyde reactants. (Reaction conditions: 10 mmol guaiacol, 5 mmol lauryl aldehyde, 150° C., 150 mg P—SiO₂. Error bars denote standard deviation from triplicates).

FIG. 8 shows a time-dependent profile of the hydroxyalkylation/alkylation (HAA) of guaiacol and lauryl aldehyde. (Reaction conditions: 10 mmol guaiacol, 5 mmol lauryl aldehyde, 100 mg P—SiO₂, 150° C. Carbon balance>90 wt. %).

FIG. 9 shows the yield of a branched benzene lubricant (BBL) and condensation byproduct at various fractional loadings of lauryl aldehyde at 2 hr (solid bars) and 4 hr (slashed bars) reaction times. (Reaction conditions: 10 mmol guaiacol, 5 mmol lauryl aldehyde (batch) 150 mg P—SiO₂, 150° C.).

FIG. 10 shows a TGA profile of fresh and spent P—SiO₂ catalyst. Heating occurs from 30 to 700° C. at a heating rate of 10 K min⁻¹ under air (30 mL min⁻¹).

FIG. 11 shows a gas chromatogram of the THF wash of the spent catalyst after the HAA reaction.

FIG. 12 shows recyclability of P—SiO₂ for the synthesis of a branched benzene lubricant. (Reaction conditions: 10 mmol guaiacol, 5 mmol lauryl aldehyde, 150 mg P—SiO₂, 150° C., 24 hr, semi-batch system).

FIG. 13 shows gas chromatogram profiles of HDO over Ir—ReOₓ catalyst at different temperatures (Reaction conditions: 0.3 g HAA reaction product, 0.2 g Ir—ReOₓ on silica, 20 ml cyclohexane, 500 rpm, 18 hr, 5 MPa H₂).

FIG. 14 shows a GCMS mass fragmentation of (a) cyclic C₂₄ alkane (1,1-dicyclohexyldodecane; and 1-cyclohexyldodecane) base oil (mass from GCMS—334.43, calculated mass—334) and (b) branched C24 alkane (11-methyl-tricosane) base oil (mass from GCMS—338.4, calculated mass—338).

FIG. 15 shows a ¹H NMR spectrum of a C₂₄ alkane lubricant mix. The sample was prepared in CDCl₃ and the predominant product was the C₂₄ cyclic alkane lubricant, followed by the C₂₄ branched alkane lubricant. The labels a-d reference the hydrogen atoms.

FIG. 16 shows a branched cyclic lubricant yield at different reaction times (Reaction conditions: 0.3 g HAA reaction product, 0.2 g Ir—ReOx on silica, 20 ml cyclohexane, 500 rpm, 200° C., 5 MPa H₂).

FIG. 17 shows gas chromatograms of the HDO of a benzene lubricant over different catalysts (Reaction conditions: 0.3 g HAA reaction product, 0.2 g catalyst, 20 ml cyclohexane, 500 rpm, 200° C., 12 hr, 5 MPa H₂).

FIG. 18 shows gas chromatograms of a HAA reaction product before (top) and after (bottom) HDO (Reaction conditions: 0.3 g HAA reaction product, 0.2 g Ir—ReOx on silica, 20 ml cyclohexane, 500 rpm, 200° C., 12 hr, 5 MPa H₂).

FIG. 19 shows an image of lubricant products. Left (clear)—cyclic and branched C₂₄ alkane base oil from the HDO reaction. Right (dark)—BBL and byproduct mixture from HAA reaction (HAA reaction conditions: 10 mmol guaiacol, 5 mmol lauryl aldehyde, 150° C., 12 h, 150 mg P—SiO₂; and HDO reaction conditions: 0.3 g HAA reaction product, 0.2 g Ir—ReOx/SiO₂ catalyst, 20 ml cyclohexane, 500 rpm, 200° C., 12 hr, 5 MPa H₂).

### DETAILED DESCRIPTION OF THE INVENTION

As used herein, the term “renewal” is used interchangeably with ““biomass-derived”, “biologically-derived”, “bio-derived” or “bio-based” and refers to compounds that are obtained from renewable resources such as plants and contain either (i) only or a substantial amount of renewable carbon or (ii) none or a minimal amount of fossil fuel-based or petroleum-based carbon. The “lignin-derived” compounds as disclosed herein refer to a chemical compound synthesized using at least one lignin monomer derived from a lignin-containing biomass, including, but not limited to, softwoods, lignocellulose biomass, solid wood waste, forest wood waste, lignin rich food waste, energy crops, animal waste, agricultural waste, or lignin residue generated by cellulosic biorefinery or paper pulping industries. Suitable lignin-rich food wastes include, but are not limited to nutshells, olive seeds, and tomato peels and seeds. Suitable energy crops include, but are not limited to, wheat, corn, soybean, sugarcane, arundo, camelina, carinate, jatropha, miscanthus, sorghum, and switchgrass. Suitable examples of lignin monomers for use in the present invention include, but are not limited to:
(i) an unsubstituted phenol or a substituted phenol, such as cresol (methyl phenol), ethylphenol, propylphenol,
(ii) a monomethoxyphenol, such as guaiacol (monomethoxy-substituted phenol), methylguaiacol, ethylguaiacol, propylguaiacol,
(iii) a dimethoxyphenol, such as a syringol (dimethoxy-substituted phenol), methyl syringol, or
(iv)combinations thereof. [Please confirm]

As used herein, the terms “syringols” and “guaiacols” refer to phenolic compounds derived from depolymerized lignins containing one phenolic hydroxyl group and in addition two methoxy groups and one methoxy group respectively. Syringols, guaiacols, and phenols can be obtained from any suitable lignin-containing biomass, including, but not limited to, softwoods, lignocellulose biomass, solid wood waste, forest wood waste, lignin rich food waste, energy crops, animal waste, agricultural waste, or lignin residue generated by cellulosic biorefinery or paper pulping industries. Suitable examples of lignin-containing biomass include, for example and without limitation, oak, alder, chestnut, ash, aspen, balsa, beech, birch, boxwood, walnut, laurel, camphor, chestnut, cherry, dogwood, elm, eucalyptus, pear, hickory, ironwood, maple, olive, poplar, sassafras, rosewood, bamboo, coconut, locust, and willow trees, as well as, but not limited to, grasses (e.g., switchgrass, bamboo, straw), cereal crops (e.g., barley, millet, wheat), agricultural residues (e.g., corn stover, bagasse), and lignin-rich food wastes (e.g., nutshells, olive seeds, and tomato peels and seeds). Syringol, guaiacol, and phenol molecules can also come from petrochemical resources.

Hence, the term “lignin-derived renewable” compositions, such as lubricant, personal care, or pharmaceutical compositions, refers to compositions comprising a compound derived from at least one lignin-derived monomer. Assessment of the amount of renewably based (bio-based) carbon in a material can be performed through standard test methods. Using radiocarbon and isotope ratio mass spectrometry analysis, the bio-based content of materials can be determined, using ASTM-D6866, a standard method established by ASTM International, formally known as the American Society for Testing and Materials.

As used herein, the “bio-based content” is determined in accordance with ASTM-D6866 and is built on the same concepts as radiocarbon dating, but without use of the age equations. The analysis is performed by deriving a ratio of the amount of radiocarbon (¹⁴C) in an unknown sample to that of a modern reference standard. The ratio is reported as a percentage with the units “pMC” (percent modern carbon) with modern or present defined as 1950. If the material being analyzed is a mixture of present day radiocarbon and fossil carbon (containing no radiocarbon), then the pMC value obtained correlates directly to the amount of biomass material present in the sample.

Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day plant/tree would give a radiocarbon signature near 107.5 pMC. If that material was diluted with 50% petroleum derivatives, it would give a radiocarbon signature near 54 pMC.

A bio-mass content result is derived by assigning 100% equal to 107.5 pMC and 0% equal to 0 pMC. In this regard, a sample measuring 99 pMC will give an equivalent bio-based content result of 93%.

Assessment of the biodegradability of a material, such as of compounds of formula (I), base oils, or compositions such as lubricant compositions and personal care compositions of the present disclosure, can be performed through standard test methods, such as those developed by the Organization for Economic Cooperation and Development (OECD), the Coordinating European Council (CEC), and the American Society for Testing and Materials (ASTM), such as, OECD 301B (the Modified Strum test), ASTM D-5864, and CEC L-33-A-934. Both OECD 301B and ASTM D-5864 measure ready biodegradability, defined as the conversion of 60% of the material to CO₂ within a ten day window following the onset of biodegradation, which must occur within 28 days of test initiation. In contrast, the CEC method tests the overall biodegradability of hydrocarbon compounds and requires 80% or greater biodegradability as measured by the infrared absorbance of extractable lipophilic compounds.

As used herein, the terms “lubricant”, “lubricant composition”, and “lubricant base oil” refer to any substance used to reduce friction by providing a protective film between two moving surfaces. In general, a lubricant exhibits one or more characteristics, such as, high viscosity index, high boiling point, thermal stability, oxidation stability, low pour point, corrosion prevention capability and low surface tension.

As used herein, a “condensation” reaction refers to a chemical reaction in which two molecules combine to form larger molecule while producing a small molecule, such as H₂O, as a byproduct.

As used herein, a “hydrogenation” reaction refers to a chemical reaction between molecular hydrogen (H₂) and an organic compound, typically in the presence of a catalyst, to reduce or saturate the organic compound.

As used herein, a “hydrodeoxygenation” or “HDO” reaction refers to a chemical reaction whereby a carbon-oxygen single bond is cleaved or undergoes lysis (cleavage of a C—O bond) by molecular hydrogen, typically in the presence of a catalyst. “HDO” is a process for removing oxygen from a compound.

The term “kinematic viscosity” is used herein to refer to a fluid's inherent resistance to flow when no external force other than gravity is acting on the fluid. “Kinematic viscosity” is measured as the ratio of absolute (or dynamic) viscosity to density.

The term “pour point” as used herein refers to the temperature below which a liquid loses its flow characteristics.

As used herein, the term “branched cycloaliphatic compound” refers to a cycloaliphatic compound having at least one alkyl group branch on the cylcloaliphatic ring. The alkyl group itself may be branched or unbranched. As used herein, the term “branched benzene lubricant (BBL)” is used interchangeably with “branched aromatic lubricant base oil,” “branched aromatic lubricant,” and “branched aromatic base oil” and refers to a composition comprising a branched aromatic compound (BAr), e.g., benzene. As used herein, the term “branched aromatic compound” refers to an aromatic compound having an alkyl group branch on the aromatic ring. The alkyl group itself may be branched or unbranched.

### Composition

According to an aspect of the present invention, disclosed herein is a bio-based composition comprising a branched aromatic compound having one of the following structures:

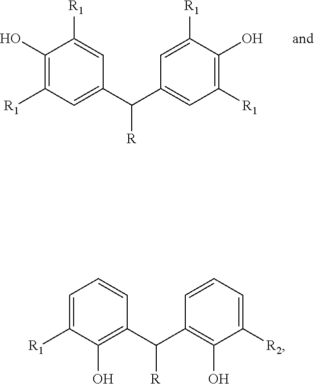

wherein R is an alkyl group having 1 to 20, or 3 to 19, or 5 to 17 carbon atoms, wherein the alkyl group is a linear, a branched, or a cycloalkyl group, and wherein R₁ and R₂ are independently selected from hydrogen and a methoxy group.

As used herein the alkyl groups may be linear, branched, or cycloalkyl group. In an embodiment, R is a linear or branched alkyl group having 1 to 20, 1-19, 1-17, 5-20, 5-19, 5-17, 10-20, 10-19, or 10-17 carbon atoms. In another embodiment, R is a cycloalkyl group having 5-20, 5-19, 5-17, 6-20, 6-19, 6-17, 7-20, 7-19, 7-17, 9-20, 9-19, 9-17, 8-20, 8-19, 8-17, 10-20, 10-19, or 10-17 carbon atoms. Suitable examples of alkyl groups include, but are not limited to, methyl group, ethyl group, any isopropyl group (e.g., an isopropyl group or a n-propyl group), any butyl group (e.g., a n-butyl group, an isobutyl group, or a sec-butyl group), any pentyl group, any hexyl group, any heptyl group, any octyl group, any nonyl group, any undecyl group, any dodecyl group (e.g. a lauryl group), any tridecyl group, tetradecyl group, any pentadecyl group, any hexadecyl group (e.g., a palmityl group), any heptadecyl group, any octadecanyl (e.g., a stearyl group), a cyclopentyl, a cyclohexyl, a cyclooctyl group, and the like.

In an embodiment, R is a branched alkyl group having 3-20, 3-19, 3-17, 5-20, 5-19, 5-17, 10-20, 10-19, or 10-17 carbon atoms. Suitable examples of branched alkyl groups, having one or more branches include, but are not limited to, isopropyl group, 2-methylpropyl group, 2-methylbutyl group, 2-methyldodecyl group, 2-ethylpropyl group, 2-ethylpentyl group, 2-ethylhexyl group, 2-ethyloctyl group, cyclopentyheptyl group, 2-propyl heptyl group, 2-butyl octyl group, 2-pentyl nonyl group , 2-hexyl decyl group, 2-heptyl undecyl group, 2-octyl dodecyl group, 2-nonyl undecyl 2-nonyl undecyl, and the like.

In an embodiment, the bio-based composition further comprises an aliphatic enal compound having the following structure:

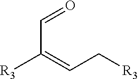

where each R₃ is independently selected from an alkyl group having 0 to 19, or 2 to 18, or 4 to 16 carbon atoms, and wherein the alkyl group is a linear, a branched, or a cycloalkyl group. In an embodiment, R₃ has one methylene group less than that present in R.

In another aspect, there is a bio-based composition comprising a branched cycloaliphatic compound having one of the following structures:

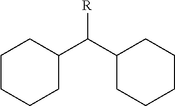

and isomers thereof, and

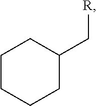

wherein R is an alkyl group having 1 to 20, or 3 to 19, or 5 to 17 carbon atoms, and wherein the alkyl group is a linear, a branched, or a cycloalkyl group.

In an embodiment, the bio-based composition further comprises a branched aliphatic compound having one of the following structures:

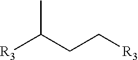

and isomers thereof
wherein each R₃ is independently selected from an alkyl group having 0 to 19, or 2 to 18, or 4 to 16 carbon atoms,
wherein the alkyl group is a linear, a branched, or a cycloalkyl group, and
wherein R₃ has one methylene group less than that present in R.

In an embodiment of the bio-based composition, as disclosed hereinabove, at least one of the one or more compounds has a bio-based content in the range of 20 to 100%, e.g., at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%; preferably 40 to 100%; and most preferably 50 to 100%, as determined according to ASTM-D6866.

Exemplary branched aromatic compounds include, but are not limited to, 2,2′-(1,1′-dodecanediyl)-6,6′-dimethoxydiphenol, 2,2′-(1,1′-dodecanediyl) diphenol, 2,2′-(1,1′-dodecanediyl)-4,4′-dimethyldiphenol, 2,2′-(1,1′-dodecanediyl)-4,4′-diethyldiphenol, 2,2′-(1,1′-dodecanediyl)-4,4′-dipropyldiphenol, 2,2′-(1,1′-dodecanediyl)-(4,4′-dimethyl)-6,6′-dimethoxydiphenol, 2,2′-(1,1′-dodecanediyl)-(4,4′-diethyl)-6,6′-dimethoxydiphenol, 2,2′-(1,1′dodecanediyl)-(4,4′-dipropyl)-6,6′-dimethoxydiphenol, 4,4′-(1,1′dodecanediyl)-2,2′,6,6′-tetramethoxydiphenol, 3,3′-(1,1′dodecanediyl)-(4,4′ dimethyl)-2,2′,6,6′-tetramethoxydiphenol.

Exemplary aliphatic enal compounds include, but are not limited to, 2-Butenal, 2-methyl-2-pentenal, 2-ethyl-2-hexenal, 2-propyl-2-heptenal, 2-(2′methyl)-ethyl-2-hetenal, 2-butyl-2-octenal, 2-pentyl-2-nonenal, 2-(2′methyl)-butyl-2-nonenal, 2-hexyl-2-decenal, 2-nonyl-2-tridecenal, 2-decyl-2-tetradecenal, 2-dodecyl-2-hexadecenal, 2-tetradecyl-2-octadecenal, 2-hexadecyl-2-eicosenal.

Exemplary branched cylcoaliphatic compounds include, but are not limited to, 1,1-dicyclohexylmethane, 1,1-dicyclohexylethane, 1,1-dicyclohexylethane, 1,1-dicyclohexylprpane, 1,1-dicyclohexyisopropoane, 1,1-dicyclohexylbutane, 1,1-dicyclohexylpentane, 1,1-dicyclohexyl-2-ethyl-pentane, 1,1-dicyclohexylhexane, 1,1-dicyclohexyl-2-ethyl-hexane, 1,1-dicyclohexylundecane, 1,1-dicyclohexyldodecane, 1,1-dicyclohexyltetradecane, 1,1-dicyclohexylhexadecane, 1,1-dicycloctadecane and isomers thereof; 1-cyclohexylmethane, 1-cyclohexylethane, 1-cyclohexylethane, 1-cyclohexylprpoane, 1-cyclohexyisopropane, 1-cyclohexylbutane, 1-cyclohexylpentane, 1-cyclohexyl-2-ethyl-pentane, 1-cyclohexylhexane, 1-cyclohexyl-2-ethyl-hexane, 1-cyclohexylundecane, 1-cyclohexyldodecane, 1-cyclohexyltetradecane, 1-cyclohexylhexadecane, 1-cycloctadecane, and the like.

Exemplary branched aliphatic compounds include, but are not limited to, Butane, 2-methyl-pentane, 2-ethyl-hexane, 2-propyl-heptane, 2-(2′methyl)-ethyl-heptane, 2-butyl-2-octenal, 2-pentyl-2-nonenal, 2-(2′methyl)butyl-nonane, 2-hexyl-decane, 2-nonyl-tridecane, 2-decyl-tetradecane, 2-dodecyl-hexadecane, 2-tetradecyl-octadecane, 2-hexadecyl-eicosane.

### Bio-Based Lubricant Composition

In an aspect, a bio-based lubricant composition comprises:
(i) a branched aromatic compound having one of the following structures:

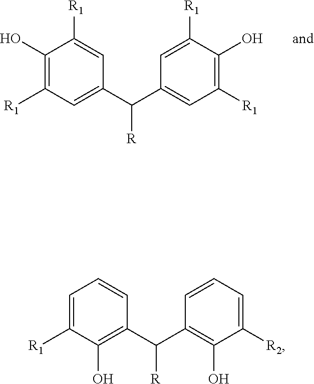

and optionally an aliphatic enal compound having the following structure:

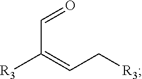

or
(ii) a branched cycloaliphatic compound having one of the following structures:

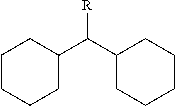

and isomers thereof, and

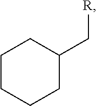

and optionally a branched aliphatic compound having the following structure:

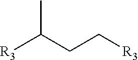

and isomers thereof,
wherein R is an alkyl group having 1 to 20, or 3 to 19, or 5 to 17 carbon atoms, wherein the alkyl group is a linear, a branched, or a cycloalkyl group, wherein R₁ and R₂ are independently selected from hydrogen and a methoxy group, and wherein each R₃ is independently selected from an alkyl group having 0 to 19, or 2 to 18, or 4 to 16 carbon atoms.

In an embodiment, the bio-based lubricant composition also comprises an effective amount of one or more lubricant additives. According to various embodiments, the bio-based lubricant composition may include one or more of (i) branched aromatic compounds, and optionally aliphatic enal compounds, and (ii) branched cycloaliphatic compounds and optionally branched aliphatic compounds, in any suitable amount, such as in an amount of at least 10%, at least 20%, at least 30%, at least 40%, at least 50%, at least 60%, at least 70%, at least 80%, at least 90%, at least 95%, at least 99% or even 100% by weight or in the range of 20 to 100%, or 50 to 99% or 75 to 99% by weight of the total weight of the lubricant composition.

In an embodiment, the bio-based lubricant composition comprises 70% to 80% of branched aromatic compounds, and 20% to 30% of aliphatic enal compound. In one embodiment, the bio-based lubricant composition is derived from guaiacol and laurylaldehyde as the reactants, and the bio-based lubricant composition comprises about 76% of branched aromatic compound (2,2′-(1,1-dodecanediyl)-6,6′-dimethoxydiphenol) and about 24% of aliphatic enal (13-decyl-12-ene-tetradecanal). In an embodiment, the bio-based lubricant composition comprises 70% to 80% of branched cycloaliphatic compounds, 20% to 30% of a branched aliphatic compound. In yet another embodiment, the bio-based lubricant composition is derived from guaiacol and laurylaldehyde as the reactants, and the bio-based lubricant composition comprises 62% branched cycloaliphatic compounds (1,1-dicyclohexyldodecane), 22% branched lubricant (11-methyl-tricosane), 12% dodecyl cyclohexane, and 4% alkanes

In an embodiment of the bio-based lubricant composition, the one or more lubricant additives may be selected from among conventional antioxidants, stabilizers, detergents, dispersants, demulsifiers, antioxidants, anti-wear additives, pour point depressants, viscosity index modifiers, friction modifiers, anti-foam additives, defoaming agents, corrosion inhibitors, wetting agents, rust inhibitors, copper passivators, metal deactivators, extreme pressure additives, and combinations thereof. Any of such lubricant additives may be used in an amount effective to impart one or more desired properties or characteristics to the lubricant composition. Typically, effective concentrations of such lubricant additives will be similar to those utilized in conventional lubricant compositions, although in certain cases lower or higher concentrations may be needed or desired due to the different characteristics of the base oils comprised of one or more branched aromatic compounds, aliphatic enal compounds, branched cycloaliphatic compounds, and branched aliphatic compounds, which are present in the lubricant compositions of the present invention. In certain cases, individual lubricant additives are included in the lubricant composition at only a few ppm, but in other cases an individual lubricant additive is employed in an amount of at least 10 ppm, at least 50 ppm, at least 100 ppm, at least 250 ppm, at least 500 ppm, at least 750 ppm, at least 1000 ppm, at least 2000 ppm, at least 3000 ppm, at least 4000 ppm, at least 5000 ppm, or even higher (e.g., at least 1% by weight), depending upon the type of lubricant additive and the effect desired to be achieved by the inclusion of the lubricant additive. Generally speaking, however, the total amount of lubricant additive does not exceed 25% by weight based on the total weight of the lubricant composition. According to other embodiments, the lubricant composition comprises not more than 20%, not more than 15%, not more than 10% or not more than 5% by weight in total of lubricant additive(s), based on the total weight of the lubricant composition.

In another embodiment, the bio-based lubricant composition may further include one or more co-base oils (i.e., base oils other than the base oil comprising one or more of (i) branched aromatic compounds, with optionally aliphatic enal compounds, and (ii) branched cycloaliphatic compounds with optionally branched aliphatic compounds. For example, the co-base oil may be selected from the group consisting of American Petroleum Institute (API) Group I base oil, Group II base oil, Group III base oil, Group IV base oil, Group V base oil, gas-to-liquid (GTL) base oil, and combinations thereof.

The American Petroleum Institute (API) has 5 base oil designations. The first three groups are derived from crude oil (mineral oil); Group IV base oils are fully synthetic; Group V is for all other base oils not included in Groups I through IV.

Group I: These base oils contain less than 90% saturates, more than 0.03% sulfur and have an SAE viscosity index range of 80 to 120. The operating temperature range is from 32 to 150 F. These oils are solvent-refined, which is a simpler refining process, making these the cheapest base oils on the market.

Group II: Group II base oils are defined as containing more than 90% saturates, less than 0.03% sulfur, and have a V.I. of 80 to 120. These base oils are often manufactured by hydrocracking, which is a more complex process than solvent-refining. These oils have better antioxidation properties and have a clearer color than Group I base oils.

Group III: These base oils contain greater than 90% saturates, less than 0.03% sulfur, and have a viscosity index above 120. Group III base oils are more refined than Group II and are typically severely hydrocracked (greater pressure and heat); this process produces a purer base oil.

Group IV: These base oils are called polyalphaolefins (PAOs). They are synthetic and made through a process called synthesizing. PAOs have a broader temperature range and therefore are preferred for use in applications exposed to extreme cold and/or high heat.

Group V: All other base oils that do not fall in the other groups are classified as Group V. Examples include silicone, phosphate esters, polyalkylene glycols (PAG), polyolesters, and biolubes. These base oils can be mixed with other base stocks to enhance an oil's properties.

According to certain embodiments, the lubricant composition comprises a) from 20 to 99.99%, or 50 to 99%, or 75 to 99% by weight of a base oil comprised of one or more of (i) branched aromatic compounds, and optionally aliphatic enal compounds, and (ii) branched cycloaliphatic compounds and optionally branched aliphatic compounds, as disclosed hereinabove, and b) from 0.01 to 80%, or 1 to 50%, or 1-25% by weight in total of one or more additional components comprising one or more lubricant additives and/or one or more co-base oils, the total of a) and b) equaling 100%.

In yet another embodiment, the lubricant composition comprising one or more of (i) branched aromatic compounds, with optionally aliphatic enal compounds, and (ii) branched cycloaliphatic compounds with optionally the branched aliphatic compounds, has a kinematic viscosity in the range of 2 to 100 Centistokes (CSt) at 100° C., preferably 2-50 CSt, most preferably 2-30 CSt and in the range of 6 to 300 CSt at 40° C., preferably 6-275 CSt, most preferably 6-250 CSt, as measured by ASTM D445, such that a viscosity index calculated from kinetic viscosity at 100° C. and 40° C., is in the range of −40 to 200, or −35 to 150, as measured by ASTM D2270, and the base oil has a kinematic viscosity of at least 3 CSt, as measured by ASTM D445.

In another embodiment, the lubricant composition, has a Noack volatility of less than 50 wt. %, or 45 wt. %, or 40 wt. %, or 35 wt. %, or 30 wt. %, or 25 wt. %, or 20 wt. %, as measured by ASTM D6375.

In an aspect of the invention, the lubricant compositions as disclosed hereinabove may be used in one or more of industrial machinery, automobiles, aviation machinery, refrigeration compressors, agricultural equipment, marine vessels, agriculture equipment, medical equipment, hydropower production machinery, and/or food processing equipment.

In another aspect of the invention, the base oil comprising one or more of (i) branched aromatic compounds, and optionally branched aliphatic compounds, and (ii) branched cycloaliphatic compounds and optionally branched aliphatic compounds, in accordance with various embodiments of the present invention, as disclosed hereinabove, may be used in one or more of industrial machinery, automobiles, aviation machinery, refrigeration compressors, agricultural equipment, marine vessels, agriculture equipment, medical equipment, hydropower production machinery, and/or food processing equipment. In another embodiment, base oil, as disclosed hereinabove, may be used in pharmaceutical formulations and personal care product formulations, e.g., sunscreen, lotion, creams, cosmetics, and the like.

According to still further embodiments, a method is provided of reducing at least one of friction or wear between a first surface and a second surface, wherein the method comprises lubricating at least one of the first surface and the second surface with a base oil or a lubricant composition comprising one or more of (i) the branched aromatic compounds, with optionally the branched aliphatic compounds, and (ii) the branched cycloaliphatic compounds with optionally the branched aliphatic compounds, in accordance with the present invention. The first surface and the second surface may be the same as or different from each other and may be constructed of any suitable material, including for example metal, coated metal, plastic, and/or ceramic.

Also provided by the present invention is a method of lowering the coefficient of friction of a substrate surface, wherein the method comprises applying a coating of a base oil or lubricant composition comprised of one or more of (i) the branched aromatic compounds, with optionally the branched aliphatic compounds, and (ii) the branched cycloaliphatic compounds with optionally the branched aliphatic compounds, as disclosed hereinabove, to the substrate surface. The substrate may be comprised of any suitable material such as metal, coated metal, plastic and/or ceramic.

In yet another aspect, a bio-based personal care composition is provided, the personal care composition including a) a base oil comprising one or more of (i) branched aromatic compounds, and optionally branched aliphatic compounds, and (ii) branched cycloaliphatic compounds and optionally branched aliphatic compounds, in accordance with various embodiments of the present invention, as disclosed hereinabove, and b) an effective amount of one or more additives. Any suitable conventional additive could be used, including, but not limited to, pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, and water. In another embodiment, the personal care composition may further comprise an active ingredient selected from the group consisting of antibiotic, antiseptic, antifungal, corticosteroid, and anti-acne agent. The personal care composition of the present disclosure may be used in any suitable application including, but not limited to, cosmetics, sunscreens, lotions, creams, antiperspirants, deodorants, and medicated ointments, creams, and oils.

In still another aspect, a bio-based pharmaceutical composition is provided, the pharmaceutical composition including a) a base oil comprising one or more of (i) branched aromatic compounds, and optionally branched aliphatic compounds, and (ii) branched cycloaliphatic compounds and optionally branched aliphatic compounds, in accordance with various embodiments of the present invention, as disclosed hereinabove, b) an effective amount of one or more pharmaceutically active ingredients, and, c) optionally, one or more pharmaceutically acceptable excipients. Any suitable pharmaceutically active ingredient(s) could be used, including, in particular, oil-soluble drugs, such as anti-inflammatory agents, antibiotics, antifungals, acne treatment agents, scabies/lice treatment agents, corticosteroids and analgesics. The pharmaceutical composition could, for example, take the form of a cream, lotion, foam, gel, ointment, emulsion (including both water-in-oil and oil-in-water emulsions) or paste and may be a topical preparation, oral formulation or injectable formulation. The base oil comprising one or more compounds of formula (I) may function, for example, as a carrier, vehicle, solubilizing excipient or filler (such as in soft gelatin capsules and the like).

### Process of Making Compounds of the Present Invention

The invention disclosed herein include processes for the preparation of a composition comprising one or more of (i) branched aromatic compounds, and optionally branched aliphatic enal compounds, and (ii) branched cycloaliphatic compounds and optionally branched aliphatic compounds, as disclosed hereinabove, and their use as base oils in lubricant compositions, pharmaceutical compositions, personal care compositions.

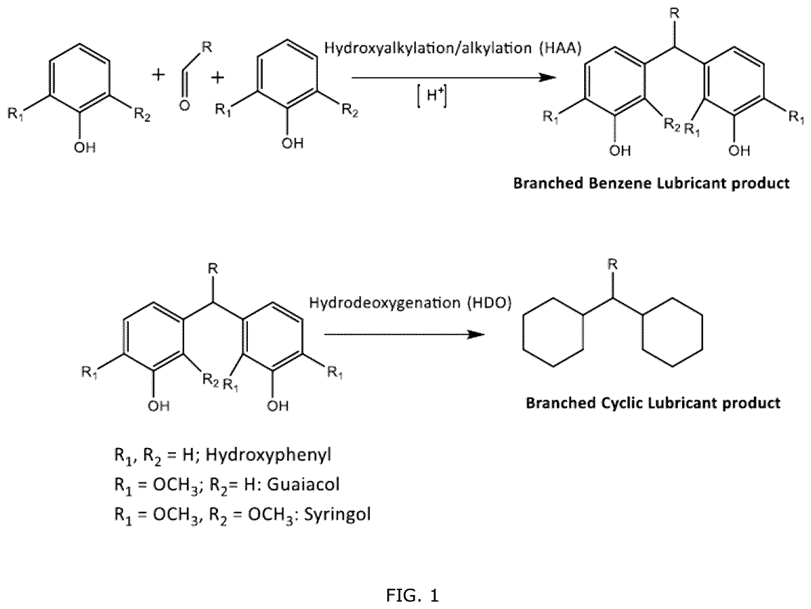

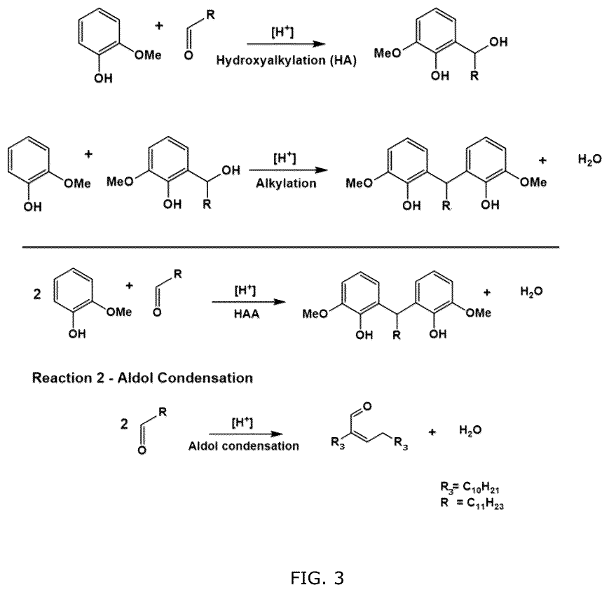

The process comprises carrying out hydroxyalkylation/alkylation (HAA) of a lignin-derived monomer containing a phenolic hydroxyl group and an aldehyde (RCHO) in the presence of an acidic catalyst, as shown in FIGS. 1 and 3, to form a bio-based branched aromatic compound having one of the following structures:

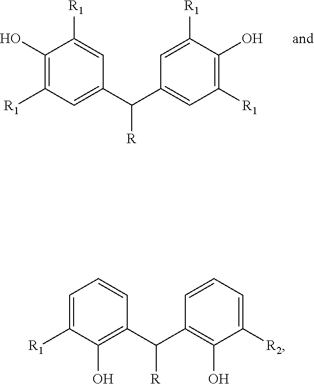

and optionally a branched aliphatic enal compound having the following structure:

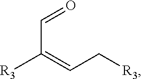

wherein R is an alkyl group having 1 to 20, or 3 to 19, or 5 to 17 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19, or 2 to 18, or 4 to 16 carbon atoms.

The lignin-derived monomer and the aldehyde may be present in any suitable amount, for example, the molar ratio of the lignin-derived monomer and the aldehyde is between 2 to 20, or 2 to 18, or 2 to 15, or 2 to 12, or 2 to 10.

Any suitable aldehyde (RCHO), dialdehyde ((CR₄R₅)ₙ(CHO)₂), enal (CHR₄═CR₅—CHO), or ketone (R₄R₅CO) may be used. In the aldehyde (RCHO), R is an alkyl group having 1 to 20, or 3 to 19, or 5 to 17 carbon atoms.

The HAA reaction can be carried out at any suitable temperature, for example in the range of 60° C. to 200° C., or 90° C. to 190° C., or 120° C. to 180° C. for any suitable amount of time such as for 2 hr to 2 days, or 4 hr to 24 hr, or 4 hr to 18 hr.

In one embodiment, the HAA is carried out in a batch process. In another embodiment, the HAA reaction is carried out in a semi-batch process, where the aldehyde is added slowly, while keeping the total amount of the aldehyde with respect to the lignin-derived monomer constant.

In an embodiment, the step of providing an aldehyde includes at least one of dehydrogenating biomass derived alcohols and selective hydrogenation of fatty acids from natural oils, waste cooking oils and/or animal fats.

Illustrative examples of suitable fatty aldehydes include, but are not limited to, C2 to C20 linear aldehydes, such as for example with even number carbon (C2, C4, C6, C8, C10, C12, C14, C16, C18, C20). Exemplary fatty aldehydes include, but are not limited to octanal, decanal, lauraldehyde, stearaldehyde, palmetaldehyde, olealdehyde and the like.

The synthesis of aldehydes of different carbon length via dehydrogenation of biomass derived alcohols (Reference 49) or selective hydrogenation of fatty acids from natural oils or WCO (Reference 50) is known in the art.

In an embodiment, the step of providing an aldehyde comprises providing an aldehyde having C1-C20 carbon atoms. In another embodiment, the aldehyde is a bio-derived fatty aldehyde. The fatty aldehyde based monomer may be derived from a fatty acid selected from the group consisting of, but not limited to, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, laccoroic acid, psyllic acid, geddic acid, ceroplastic acid, hexatriacontylic acid, ricinoleic acid, the unsaturated analogues of these acids, the saturated and unsaturated alcohols analogous to these acids (which may be obtained, for example, by reduction of the carboxylic acid group present in a fatty acid to an aldehyde group or by conversion of a triglyceride to fatty acid methyl esters and reduction of the fatty acid methyl esters to fatty alcohols), and combinations thereof.

Fatty aldehydes may also be prepared by dehydrogenation of fatty alcohols which are derived from triglycerides from animal fats, oils, or waxes; vegetable fats, oils, or waxes [e.g., soy oil, linseed oil, rapeseed (canola) oil, cottonseed oil, olive oil, corn oil, fish oil, sunflower oil, canola oil, peanut oil, coconut oil, castor oil, jatropha oil, laurel oil, palm oil, palm kernel oil, and sesame oil]; or combinations thereof. Suitable examples of biomass derived alcohols include, but are not limited to, ethanol, butanol, hexanol, and dodecanol. Such biomass derived alcohols may be derived from any suitable biomass including, but not limited to, corn grain, soya bean grain, any kind of hard wood, any kind of soft wood, and algae. Suitable examples of fatty acids include, but are not limited to lauric acid and steric acid. Such fatty acid may be derived from any suitable natural cooking oils including, but not limited, to coconut oil, palm oil, rapeseed oil, vegetable oil, corn oil, peanut oil, olive oil, canola oil, and sunflower oil.

Any suitable lignin-derived monomer may be used, including, but not limited to:
(i) an unsubstituted phenol or a substituted phenol, such as cresol (methyl phenol), ethylphenol, propylphenol,
(ii) a monomethoxyphenol, such as guaiacol (monomethoxy-substituted phenol), methylguaiacol, ethylguaiacol, propylguaiacol,
(iii) a dimethoxyphenol, such as a syringol (dimethoxy-substituted phenol), methyl syringol, or
(iv)combinations thereof.

The hydroxyalkylation/alkylation (HAA) acidic catalyst can be, for example, any suitable liquid acid including inorganic liquid acids and organic liquid acids, or any suitable solid acid. Exemplary liquid acids include, but are not limited to a homogeneous catalyst comprising sulfuric acid, methanesulfonic acid, acetic acid, triflic acid, or p-toluenesulfonic acid. Suitable solid acid catalysts include, but are not limited to perfluorinated sulfonic acid resins, sulfonic-acid-functionalized cross-lined polystyrene resins, zeolites, or silica supported H₃PO₄. Exemplary solid acids include, but are not limited to, Amberlyst® resins (e.g. Amberlyst®-15, Amberlyst®-36), Nafion® resins (e.g., Nafion® NR50), Aquivion® Resins (e.g., Aquivion® PW98, Aquivion® PW79S), Zeolites (e.g. ZSM-5, HBEA, HY), and silica supported H₃PO₄.

The process further comprises hydrodeoxygenating (HDO) the branched aromatic compound in the presence of a hydrodeoxygenation (HDO) catalyst to obtain a cycloaliphatic compound having the following formula:

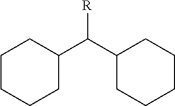

wherein R is an alkyl group having 1 to 20, or 3 to 19, or 5 to 17 carbon atoms, and wherein the alkyl group is a linear, a branched, or a cycloalkyl group.

Any suitable HDO catalyst may be used, such as a solid acid supported metal based catalyst or a physical mixture of a metal based catalyst, preferably Pd/C, Pd/SiO₂ and Pt/C, with a solid acid. Suitable solid acid supported metal based catalysts include, but are not limited to, Ni/ZSM-5, Pd/ZSM-5, Pd/BEA; a physical mixture of a metal based catalyst with a solid acid, which includes, but is not limited to, Pd/C+ZSM-5, Pd/C+BEA, Pt/C+BEA, and preferably supported metal-metal oxide catalysts such as Ir—ReOₓ/SiO₂, Ir—MoOₓ/SiO₂ or ¹M²MO/SiO₂, where ¹M can be chosen from among Ir, Ru, Ni, Co, Pd, Pt, Rh and ²M can be chosen from among Re, Mo, W, Nb, Mn, V, Ce, Cr, Zn, Co, Y, Al).

Thus, the present invention provides a novel strategy to synthesize new and existing lubricant base oils with structural diversity and tunable properties using energy efficient C—C coupling and commonly used refinery methods without complex separations that are necessary for current petroleum-based base-oils. Non-food biomass and natural or waste cooking oils can be harnessed to obtain lignin-derived monomers and aldehydes of varying carbon length and branching, possessing versatile chemistry to provide opportunities to build base oils molecules of varying structural features and properties for a wide range of targeted applications. The use of efficient and easily separable heterogeneous catalysts, as opposed to corrosive homogeneous acid catalysts currently used for synthetic base oils synthesis, enables high products selectivity and yield. In addition, low temperature processing compared to the current refinery processing (cracking and distillation for synthetic and mineral base oils), and the use of sustainable feedstock with abundant supply and possible biodegradability could make this process and products competitive and adaptive to the existing market place. Unique properties of bio-based lubricant compositions are appealing for exploring their market potential and application segments. The lubricant compositions comprising branched aromatic compounds and the branched cycloaliphatic compounds, have comparable or better properties, compared to current commercial mineral or synthetic base oils, have the potential to revolutionize out-of-box thinking for the synthesis of commercial relevant base-oils and replacement of current synthetic base-oils that have challenges associated with selectivity, separations, tuning molecular structures for desired properties etc. The properties can be predicted by molecular simulation to inform the design of molecules, an approach previously unavailable for petroleum-derived base oils that cannot be synthesized with molecular specificity.

### Aspects of the Invention

Certain illustrative, non-limiting aspects of the invention may be summarized as follows:

Aspect 1. A bio-based composition comprising a branched aromatic compound having one of the following structures:

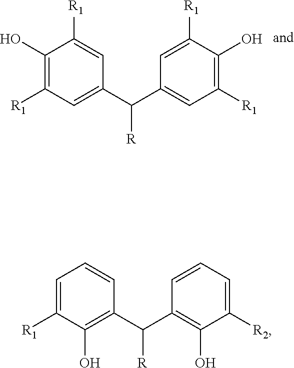

wherein R is an alkyl group having 1 to 20, or 3 to 19, or 5 to 17 carbon atoms, wherein the alkyl group is a linear, a branched, or a cycloalkyl group, and wherein R₁ and R₂ are independently selected from hydrogen and a methoxy group. Aspect 2. The bio-based composition of Aspect 1, further comprising a bio-based aliphatic enal compound having the following structure:

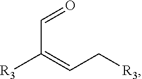

where each R₃ is independently selected from an alkyl group having 0 to 19, or 2 to 18, or 4 to 16 carbon atoms, and wherein the alkyl group is a linear, a branched, or a cycloalkyl group.

Aspect 3. A bio-based composition comprising a branched cycloaliphatic compound having one of the following structures:

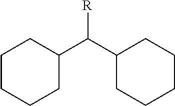

and isomers thereof, and

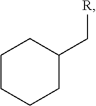

wherein R is an alkyl group having 1 to 20, or 3 to 19, or 5 to 17 carbon atoms, and wherein the alkyl group is a linear, a branched, or a cycloalkyl group.

Aspect 4. The bio-based composition of Aspect 3, further comprising a branched aliphatic compound having the following formula:

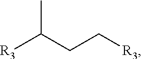

and isomers thereof,
wherein each R₃ is independently selected from an alkyl group having 0 to 19, or 2 to 18, or 4 to 16 carbon atoms, and wherein the alkyl group is a linear, a branched, or a cycloalkyl group.

Aspect 5. The bio-based composition according to any one of Aspects 1-4, wherein at least one of the compounds has a bio-based content in the range of 20 to 100%, according to ASTM-D6866.

Aspect 6. A bio-based lubricant composition comprising:
(i) a branched aromatic compound according to Aspect 1, and optionally an aliphatic enal compound of Aspect 2; or
(ii) a branched cycloaliphatic compound of Aspect 3, and optionally a branched aliphatic compound of Aspect 4; and
(iii) an effective amount of one or more additives.

Aspect 7. The bio-based lubricant composition according to Aspect 6, wherein the one or more additives are selected from the group consisting of antioxidants, stabilizers, detergents, dispersants, demulsifiers, antioxidants, anti-wear additives, pour point depressants, viscosity index modifiers, friction modifiers, anti-foam additives, defoaming agents, corrosion inhibitors, wetting agents, rust inhibitors, copper passivators, metal deactivators, extreme pressure additives, and combinations thereof.

Aspect 8. The bio-based lubricant composition according to Aspect 6 or Aspect 7, further comprising one or more co-base oils selected from the group consisting of API Group I base oil, Group II base oil, Group III base oil, Group IV base oil, Group V base oil, gas-to-liquid (GTL) base oil, and combinations thereof.

Aspect 9. The bio-based lubricant composition according to any one of Aspects 6-8, wherein the lubricant composition has a kinematic viscosity at 100° C. in the range of 2 to 100 CSt, as measured by ASTM D445.

Aspect 10. The bio-based lubricant composition according to any one of Aspects 6-9, wherein the lubricant composition has a kinematic viscosity at 40° C. in the range of 5 to 300 CSt, as measured by ASTM D445.

Aspect 11. The bio-based lubricant composition according to any one of Aspects 6-10, wherein the lubricant composition has a viscosity index calculated from kinetic viscosity at 100° C. and 40° C., in the range of −40 to 200, as measured by ASTM D2270.

Aspect 12. Use of the bio-based lubricant composition according to any one of Aspects 6-11, in one or more of industrial machinery, automobiles, aviation machinery, refrigeration compressors, agricultural equipment, marine vessels, medical equipment, hydropower production machinery, and food processing equipment.

Aspect 13. A method comprising carrying out hydroxyalkylation/alkylation of a lignin-derived monomer containing a phenolic hydroxyl group and an aldehyde (RCHO) in the presence of an acidic catalyst to form a bio-based branched aromatic compound according to Aspect 1, and optionally a branched aliphatic enal compound according to Aspect 2.

Aspect 14. The method according to Aspect 13, wherein the lignin-derived monomer comprises:
(i) an unsubstituted phenol or a substituted phenol, such as cresol (methyl phenol), ethylphenol, propylphenol,
(ii) a monomethoxyphenol, such as guaiacol (monomethoxy-substituted phenol), methylguaiacol, ethylguaiacol, propylguaiacol,
(iii) a dimethoxyphenol, such as a syringol (dimethoxy-substituted phenol), methyl syringol, or
(iv) combinations thereof.

Aspect 15. The method according to Aspect 13 or Aspect 14 further comprising hydrodeoxygenating the bio-based branched aromatic compound in the presence of a hydrodeoxygenation catalyst to obtain a bio-based cycloaliphatic compounds according to Aspect 3.

Aspect 16. The method according to any one of Aspects 13-15, wherein the hydroxyalkylation/alkylation acidic catalyst is a homogeneous catalyst comprising sulfuric acid, methanesulfonic acid, acetic acid, triflic acid, or p-toluenesulfonic acid.

Aspect 17. The method according to any one of Aspects 13-15, wherein the hydroxyalkylation/alkylation acidic catalyst is a solid acid catalyst comprising perfluorinated sulfonic acid resins, sulfonic acid-functionalized cross-lined polystyrene resins, zeolites, or silica supported H₃PO₄.

Aspect 18. The method according to Aspect 15, wherein the hydrodeoxygenation catalyst is a solid acid supported metal-based catalyst selected from Ni/ZSM-5, Pd/ZSM-5, Pd/BEA, or a physical mixture of a metal based catalyst with a solid acid, including Pd/C+ZSM-5, Pd/C+BEA, Pt/C+BEA, Pt-WOx/C and preferably a supported metal-metal oxide catalyst.

Aspect 19. The method according to Aspect 18, wherein the supported metal-metal oxide catalyst comprises Ir—ReOₓ/SiO₂, Ir—MoOₓ/SiO₂ or ¹M²MO/SiO₂, wherein ¹M =Ir, Ru, Ni, Co, Pd, Pt, or Rh and ²M=Re, Mo, W, Nb, Mn, V, Ce, Cr, Zn, Co, Y, or Al.

Aspect 20. Use of the compounds prepared according to any one of claims 13-19, as a base oil in pharmaceutical and personal care products.

Aspect 21.A personal care composition comprising:
a) a base oil comprising one or more of the following:
   (i) a branched aromatic compound according to Aspect 1, and optionally an aliphatic enal compound according to Aspect 2; or
   (ii) a branched cycloaliphatic compound according to Aspect 3, and optionally a branched aliphatic compound according to Aspect 4;
b) an effective amount of one or more additives selected from the group consisting of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, and water; and
c) optionally, an active ingredient selected from the group consisting of antibiotic, antiseptic, antifungal, corticosteroid, and anti-acne agent.

Aspect 22.A pharmaceutical composition comprising:
(a) a base oil comprising one or more of the following:
   (i) a branched aromatic compound according to Aspect 1, and optionally an aliphatic enal compound according to Aspect 2; or
   (ii) a branched cycloaliphatic compound according to Aspect 3, and optionally a branched aliphatic compound according to Aspect 4;
(b) an effective amount of one or more pharmaceutically active ingredients; and
(c) optionally, one or more pharmaceutically acceptable excipients.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The term “about” refers to the variation in the numerical value of a measurement, e.g., temperature, weight, percentage, length, concentration, and the like, due to typical error rates of the device used to obtain that measure. In one embodiment, the term “about” means within 5% of the reported numerical value.

As used herein, the singular form of a word includes the plural, and vice versa, unless the context clearly dictates otherwise. Thus, the references “a”, “an”, and “the” are generally inclusive of the plurals of the respective terms. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of”.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the compounds for use as a lubricant base oil, lubricant base oil compositions based on such compounds and process for making such compounds. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

Examples of the present invention will now be described. The technical scope of the present invention is not limited to the examples described below.

### Abbreviations

The meaning of abbreviations is as follows: “cm” means centimeter(s), “g” means gram(s), “h” or “hr” means hour(s), “HPLC” means high pressure liquid chromatography, “m” means meter(s), “min” means minute(s), “mL” means milliliter(s), “mm” means millimeter(s), “MPa” means megapascal(s), “psi” means pound(s) per square inch, “rpm” means revolutions per minute, “wt %” means weight percent(age).

### General Materials and Methods

### Materials

Aquivion PW98 ([coarse powder, Brunauer-Emmett-Teller (BET) surface area <1 m²/g, and 1.0 mmol H⁺/g), Nafion NR50 (pellets, BET surface area <1 m²/g, and 0.89 mmol H⁺/g), Amberlyst-15 (dry hydrogen form; pore size, 34.3 nm; BET surface area, 42 m²/g; and 4.8 mmol H⁺/g), Pd/C (10 wt % Pd loading), and sulphuric acid were purchased from Sigma-Aldrich. Amberlyst-36 dry resin (pore size, 32.9 nm; BET surface area, 33 m²/g; and 5.4 mmol H⁺/g) was purchased from the Rohm and Haas Company. Zeolite Hβ 12.5 (CP814e, Si/Al=12.5) was purchased from Zeolyst and calcined before reaction. Silica gel (Fuji Silysia G-6) was purchased from Fuji silica. Silica gel (high-purity grade, pore size 60 Å, 70-230 mesh) and H₂IrCl₆ containing 2.0976 wt. % Iridium (Ir) were purchased from Sigma Aldrich, NH₄ReO₄ was purchased from Alfa Aesar, and Phosphoric acid (85 wt. %) was purchased from Fisher chemical.

Guaiacol (≥98.0%), 4-methyl guaiacol (≥98.0%), 4-ethyl guaiacol (≥98.0%), 4-propyl guaiacol (≥99%), 2,6-dimethoxyphenol (99%), 4-methyl-2,6-dimethoxyphenol (97+%), phenol (99.0-100.5%), p-cresol (≥99%), 4-ethylphenol (99%), 4-propylphenol (99%), lauryl aldehyde (≥95%), and hexadecane (99%), were purchased from Sigma-Aldrich. Cyclohexane (99.9%) was purchased from Fisher Chemical.

### Catalyst Preparation

Certain of the processes described in the examples required the use of catalysts; namely, a P—SiO₂ catalyst or an Ir—ReOₓ/SiO₂ catalyst.

The P—SiO₂ catalyst (H₃PO₄, 10 wt % loading) was prepared by the impregnation method. First, SiO₂ (high-purity grade, pore size 60 Å, 70-230 mesh, Sigma-Aldrich) was impregnated with an aqueous H₃PO₄ solution. After evaporating the solvent at 75° C. on a hotplate and subsequently drying at 110° C. for 12 hr in an oven, the fine powder catalyst was calcined in a crucible in air at 500° C. for 3 hr with a 2° C./min temperature ramp.

The Ir—ReOₓ/SiO₂ (Ir, 4 wt. %; Re/Ir=2) catalyst was prepared using sequential impregnation. First, Ir/SiO₂ was prepared by impregnating Ir on calcined SiO₂ (Fuji Silysia G-6, average pore diameter 6 nm, pore volume 0.7 ml/g) using 2-3 drops of an aqueous solution of 63 wt. % H₂IrCl₆, mixing with a glass rod and crushing the lumps and drying. The process was repeated until all the aqueous solution of 63 wt. % H₂IrCl₆ was exhausted. After evaporating the solvent at 75° C. on a hotplate for 2 hours (crushing the lumps every 15 minutes) and drying at 110° C. for 12 hr in an oven, the resulting Ir/SiO₂ was impregnated with ReOₓ using 2-3 drops of an aqueous solution of 3.7 wt. % NH₄ReO₄, mixing with a glass rod and crushing the lumps and drying. The process was repeated until all the aqueous solution of 3.7 wt. % NH₄ReO₄ was exhausted. After evaporating the solvent at 75° C. on a hotplate for 2 hours (crushing the lumps every 15 minutes) and drying at 110° C. for 12 hr in an oven, the catalyst was calcined in a crucible in air at 500° C. for 3 hr with a 10° C./min temperature ramp. The 4 wt. % WOx-Pt/C was prepared using the wet impregnation method. The reported metal loadings are based on the theoretical amount of metals in the precursor solution used in impregnation.

### Reaction Procedures for Catalysis

Process of Making a Bio-Based Branched Aromatic (BAr) Compound by Hydroxyalkylation/Alkylation (HAA) Reaction

HAA reactions were performed in a Q-Tube™ Pressure Tube (Sigma Aldrich) reactor (hereinafter “q-tube vial”). In a typical reaction, 10 mmol (1.24 g) guaiacol and 5 mmol (0.92 g) lauryl aldehyde (without any solvent) were mixed in a 12-ml glass q-tube vial. The vial was placed in a preheated heating block and stirred at 500 rpm using a magnetic bar on a stirring hotplate. In the last step, the catalyst was added to the q-tube vial, and the reaction continued at the desired temperature for a specified reaction period. After the reaction, the q-tube vial was cooled in an ice bath, and the solution was diluted using 10 ml of cyclohexane solvent. A small amount of hexadecane was added as an internal standard. For the recycle experiment, the recovered catalyst was washed thrice with cyclohexane and dried in air overnight. The spent (washed) catalyst was regenerated by calcination in air at 500° C. for 3 hr at a heating rate of 10° C. min⁻¹ before reuse.

For the semi-batch reactions, 1.25 mmol of lauryl aldehyde was added every 4 hr through an inlet valve connected to a syringe into the glass vial. This procedure was performed 4 times, yielding a total amount of 5 mmol, similar to the total lauryl aldehyde amount in the batch system.

The reaction mixture was transferred to a 20 ml centrifuge vial and centrifuged at 13500×g for 5 min at 5° C. The reaction mixture was then decanted into a new reactor with a fresh catalyst, and the deactivated (spent) P—SiO₂ catalyst was isolated for regeneration and thermogravimetric characterization.

### Process of Making a Bio-Based Branched Cycloaliphatic Compound (BCA) by Hydrodeoxygenation (HDO) Reaction

HDO of BArs over Ir—ReOx/SiO₂ (and other catalysts) was performed in a 50-ml batch reactor (4790 pressure vessel, Parr Instrument Company) with an inserted Teflon liner and a magnetic stirrer. First, we added the catalyst (0.2 g) and solvent (10 ml of cyclohexane) to the reactor for catalyst prereduction and sealed the reactor with the reactor head equipped with a thermocouple, a rupture disk, a pressure gauge, and a gas release valve. The mixture was heated at 200° C. and 5 MPa H₂ for 1 hr at 240 rpm. Upon catalyst reduction, the reactor was cooled to room temperature, and the pressurized gases in the headspace were released. Then, we added BArs (0.3 g) in a fume hood, closed the reactor head immediately, purged the reactor with 1 MPa H₂ three times, pressurized to 5 MPa H₂, and heated it to the desired temperature under continuous stirring at 500 rpm. The set temperature was reached in about 25 min with a total pressure of ˜6 MPa. Upon reaction, the reactor was immediately transferred to an ice bath. The reaction solution was diluted using 15 ml of cyclohexane with a small amount of hexadecane as an internal standard, and the catalyst was separated from the solution by filtration.

### Analysis of Products

The HAA products starting from guaiacol and lauryl aldehyde included the following branched aromatic (BAr) and aliphatic enal compounds:
BBL: 2,2′-(1,1-dodecanediyl)-6,6′-dimethoxydiphenol
Aldol condensation product: 13-decyl-12-ene-tetradecanal

The HDO products included the following branched cycloaliphatic compounds (BCA) and branched aliphatic compounds:
BCL: 1,1-dicyclohexyldodecane

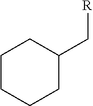

1-cyclohexyldodecane
Branched C24 alkane: 11-methyl-tricosane

As used herein, the terms “BAr” is used interchangeably with “BBL” and the term “BCA” is used interchangeably with “BCL.”

The products (BBL and BCL) were analyzed using a gas chromatograph (GC, Agilent 7890A) equipped with an HP-1 column and a flame ionization detector using hexadecane (C₁₆) as an internal standard. The products were identified by a GC (Agilent 7890B) mass spectrometer (MS) (Agilent 5977A with a triple-axis detector) equipped with a DB-5 column, high-resolution MS with liquid injection field desorption ionization, ¹H nuclear magnetic resonance (NMR) (Bruker AV400, CDCl₃ solvent).

The conversion and the yield of all products were calculated on a carbon molar basis as follows:

$$\\ \begin{matrix}Conversion [ \% ] = \frac{moles of initial reactant - moles of unreacted reactant}{moles of initial reactant} \times 100 & ( 1 )\end{matrix}$$

$$\\ \begin{matrix}Yield of detected products [ {\%}_{- C} ] = \frac{{moles}_{product} \times C atoms in product}{\begin{matrix}moles of total C atoms of guaiacol + \\ lauryl aldehyde reactants\end{matrix}} \times 100 & ( 2 )\end{matrix}$$

The selectivity to each product is defined as follows.

$$\\ \begin{matrix}{Selectivity}_{BBL} [ \% ] = \frac{{Yield}_{BBL}}{{Yield}_{BBL} + {Yield}_{Enal byproduct}} \times 100 & ( 3 )\end{matrix}$$

The reaction rates for forming BAR and enal byproduct were obtained at temperatures of 130, 140, and 150° C. These values were then fit using an Arrhenius plot to obtain the apparent activation energy (Eₐ) for the products.

### Thermogravimetric Analysis (TGA)

TGA was performed on fresh and spent P—SiO₂ catalysts using a TA instruments Q600 SDT thermogravimetric analyzer and differential scanning calorimeter (DSC) using a temperature program of 30 to 700° C. at a heating rate of 10 K min⁻¹ under air (30 mL min⁻¹).

### Gas Composition Analysis

The gas component was analyzed using a MicroGC (990, Agilent) with a thermal conductivity detector (TCD). The MicroGC is equipped with four columns: 1 MS5A column with Ar carrier, 1 MS5A column, 1 PPU column, and 1 PPQ column with He carrier. The setup enables the detection of H₂, He, N₂, CH₄, C₂H₆, C₂H₄, C₃H₆, C₃H₈, C₄H₈, CO, CO₂, and O₂ gases. N₂ gas was used to purge the MS detector for 20 min before feeding the sample.

### Lubricant Properties

For the branched cycloaliphatic (BCA) lubricant product, cyclohexane was removed by rotary evaporation prior to viscosity measurements. The HAA product, containing branched aromatic (BAr) compound and enal byproduct, was characterized as-is (without further treatment). The properties of the synthesized bio-based oils were evaluated according to the American Society for Testing and Materials (ASTM) methods. The kinematic viscosities at 100° and 40° C. (KV₁₀₀ and KV₄₀) were determined using the ASTM D445 method. The viscosity of the BCA product was measured using an extra-low-charge semi-micro viscometer (Cannon, size 150, calibrated model #: 9722-H62, calibrated with Cannon N35 Standard) apparatus. The sample charge volume was 300 μL. The VI was calculated using the KV₁₀₀ and KV₄₀ following the ASTM D2270 method. All other measurements were performed at the Southwest Research Institute in San Antonio, Texas, USA. The Noack volatility was measured according to the ASTM D6375 method.

### Catalyst Screening

Initial screening of different acid catalysts was conducted using guaiacol with lauryl aldehyde as the starting substrates, as shown in FIG. 1 (Reaction conditions: 10 mmol guaiacol, 5 mmol lauryl aldehyde, 8 hr, 150° C., 0.05 mmol H⁺). Various catalysts used in the screening included sulfonic acid resins, e.g., perfluorinated sulfonic acid resins (Aquivion® PW98, Nafion™ NR50), sulfonic acid-functionalized crosslinked polystyrene resins (Amberlyst™-15 and Amberlyst™-36), commercial HB zeolite (CP814e), sulphuric acid, and phosphorous on silica (P—SiO₂) at equivalent acid amounts.

Any suitable synthetic route can be used to prepare guaiacol from lignins, such as those described in Wang, S. et al. ACS Cent. Sci. 2018, 4 (6), 701-708; Ebikade, O. E. et al. Green Chem. 2020, 22, 7435-7447; Sadula, S. et al. Green Chem. 2021, 23, 1200-1211; and Ebikade, O. E., “Catalysis and Process Engineering for Unconventional Biomass Conversion,” Doctoral dissertation, University of Delaware, 2021, the entire disclosures of which are incorporated herein by reference. Lauraldehyde can be synthesized from lauric acid of coconut oil by selective hydrogenation (Yokoyama et al., Appl. Catal. A: Gen. 221, 227-239 (2001).

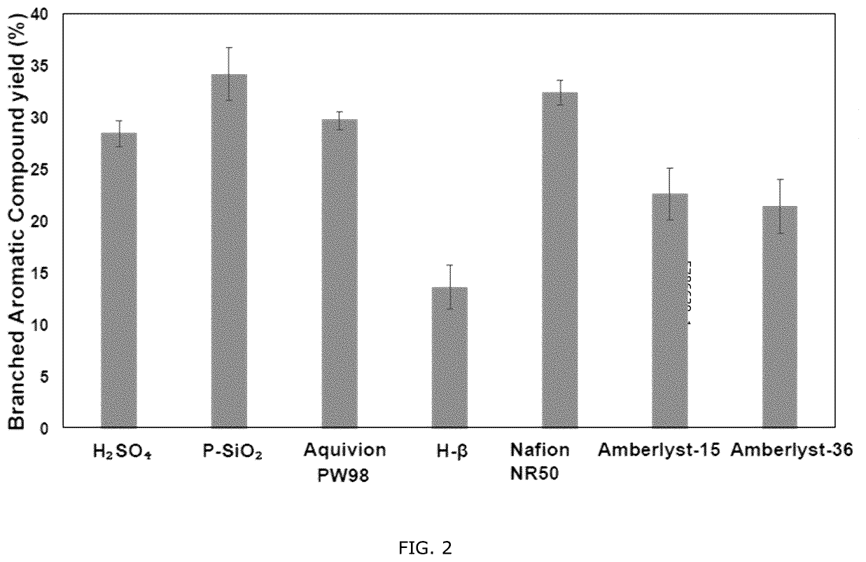

FIG. 2 shows the catalytic performance (evaluated based on the branched aromatic compound (BBL) yield) follows the order of P—SiO₂>Nafion NR50>Aquivion PW98>H2SO4>Amberlyst-15>Amberlyst-36>H−β. Without wishing to be bound by any particular theory, it is believed that mesoporosity of P-SiO2 minimizes diffusion limitations expected for large molecules in microporous zeolites, coupled with a moderate acid strength.

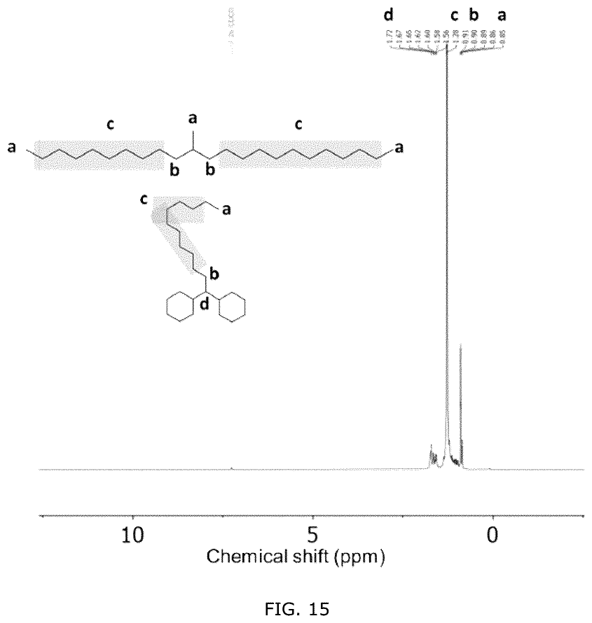

It should be noted that the mild HAA reaction conditions (65° C., 6.5 hr, 50 mg P—SiO₂) used for the HAA of furan and aldehydes resulted in no products with a guaiacol feedstock (Liu et al., Sci. Adv., 2019, 5, 1-8). This is attributed to the electron-donation of the oxygen lone pair of electrons and excessive n electron density of the furan ring that makes it more reactive, as discussed in References 30-33, especially for reactions involving an electrophilic aromatic substitution. Auto-condensation of the aldehyde was also observed, as reported elsewhere (References 6, 25, 34-36), as shown in FIG. 3. The enal product has the same number of carbons as the BAr and could also be used as a lubricant base oil. The GC-MS detected fragments at m/z=414 and m/z=350.4 (FIG. 14) correspond to the BAR and aldol condensation enal product and their geometric isomers (e.g., meta-meta, meta-ortho, ortho-ortho dimers). ¹H NMR spectra (FIG. 15) confirmed the reaction products identified via the GC-MS. All isomers of the same m/z fragment were lumped into a single product for yield calculations. Among the catalysts tested, P-SiO2 gives the highest BAr yield and is amenable to regeneration, and was selected for the remaining of the work reported below.

### Effect of the P—SiO₂ Catalyst Amount, Temperature and Reaction Time on the HAA Chemistry

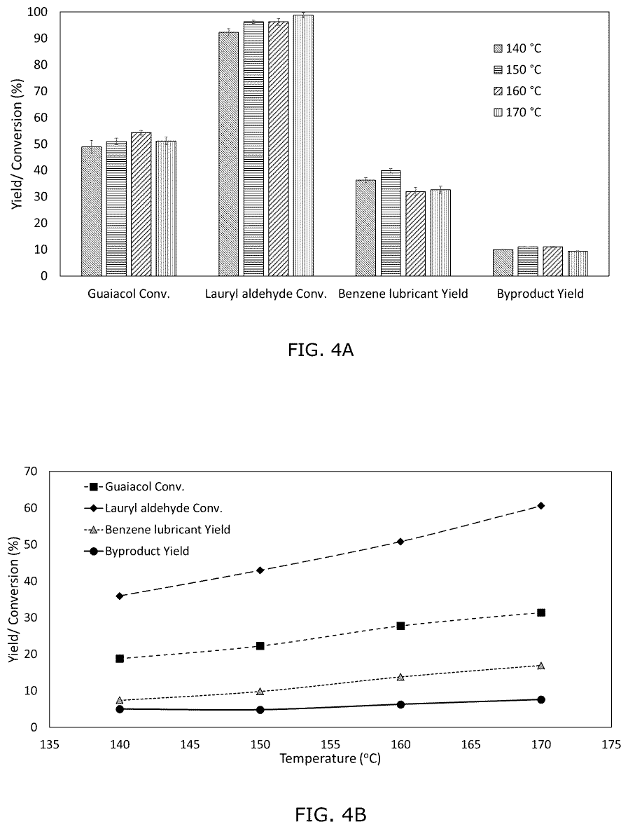

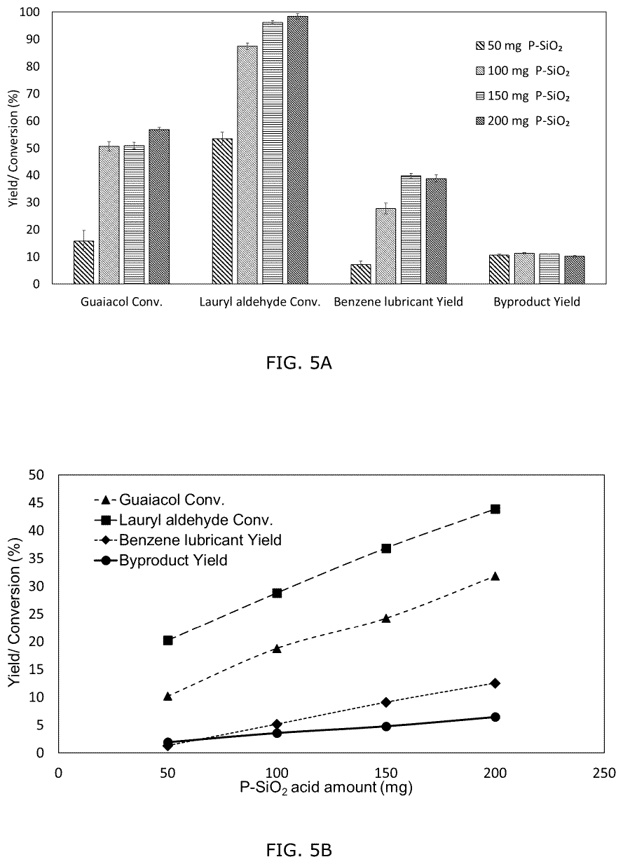

Next, the effect of varying the P—SiO₂ amount, temperature, and reaction time on the HAA chemistry was investigated. A clear dependence on the reaction temperature was observed, as shown in FIG. 4A (Reaction conditions: 10 mmol guaiacol, 5 mmol lauryl aldehyde, 12 hr, 150 mg P—SiO₂) and FIG. 4B (Reaction conditions: 10 mmol guaiacol, 5 mmol lauryl aldehyde, 1 hr, 150 mg P—SiO₂), with 150° C. giving the highest BAr yield. Above 150° C., there is no significant gain in lubricant comprising BAr yield at high conversions of reactants, as shown in FIG. 4A. FIGS. 5A (Reaction conditions: 10 mmol guaiacol, 5 mmol lauryl aldehyde, 12 hr, 150° C.) and 5B (Reaction conditions: 10 mmol guaiacol, 5 mmol lauryl aldehyde, 1 hr, 150° C.) show the increased conversions of guaiacol and lauryl aldehyde and the yield of BAR and enal byproduct with increasing the amount of P—SiO₂, as expected and shown in FIG. 3.

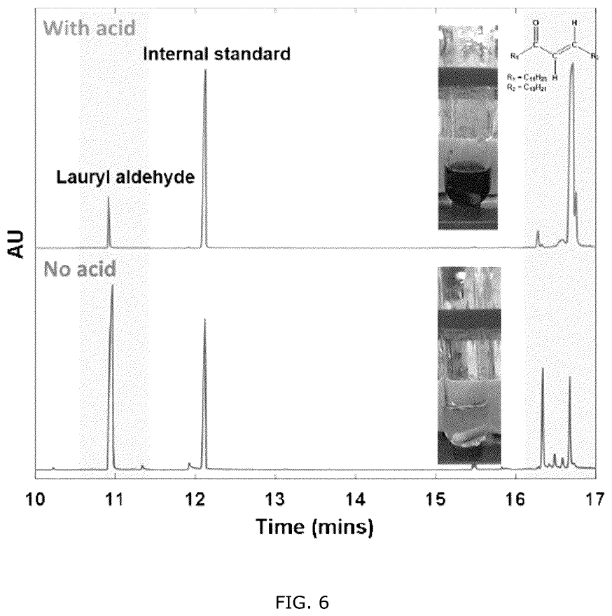

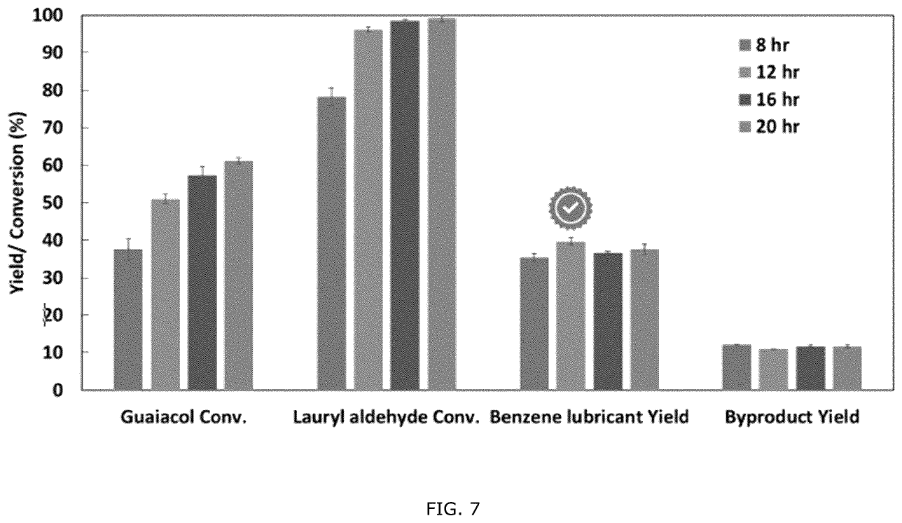

Heating lauryl aldehyde alone at 150° C. for 15 hr without P—SiO₂ catalyst led to enal product formation with 37% of lauryl aldehyde conversion resulting in a light-yellow solution, as shown in FIG. 6 (Reaction conditions: 5 mmol lauryl aldehyde, 100 mg P—SiO₂, 150° C., 15 hr). Upon adding the P—SiO₂ acid catalyst and repeating this experiment, almost all, about 98% lauryl aldehyde was converted into the enal product, forming a dark-brown solution, as shown in FIG. 6, further illustrating that the P—SiO₂ catalyzes the aldol condensation reaction, as shown in FIG. 3. These results indicate that the condensation reactions can be driven solely by high temperature and long reaction times. Furthermore, condensation is exacerbated in the presence of the acid catalyst, where almost all lauryl aldehyde is converted into condensation products. Hence, maybe diluting the reactants in a solvent for the HAA reaction could minimize side reactions and increase lubricant yield, thereby achieving a higher BAr yield by reducing the side reactions. Above a P—SiO₂ loading of 150 mg, no significant increase in BAR yield, guaiacol, and lauryl aldehyde conversion is seen from FIG. 5A. A 12 hr reaction time gave the highest BAr yield, as shown in FIG. 7 (Reaction conditions: 10 mmol guaiacol, 5 mmol lauryl aldehyde, 150° C., 150 mg P—SiO₂. Error bars denote standard deviation from triplicates).

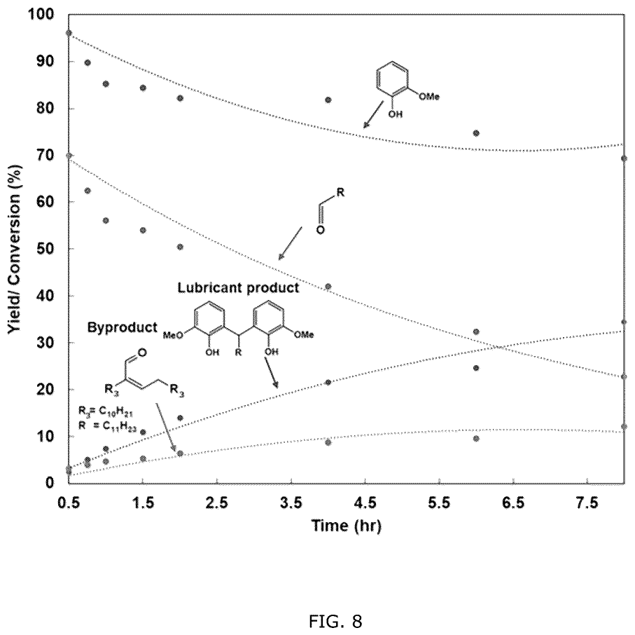

Due to the relatively constant yield of BAR at the reaction times depicted in FIG. 7, a time-dependent study at shorter reaction times was performed to understand the primary and secondary reactions taking place, as shown in FIG. 8. Both HAA and aldol condensation reactions occur in parallel, affording BAr and enal product, respectively. Simultaneous lauryl aldehyde consumption in the parallel HAA and aldol condensation reactions explains its steeper consumption than guaiacol in FIG. 8. The initial reaction rate data shown in Table 1 (Table S1) at various temperatures was used to estimate the apparent activation energies (Eₐ) for Reaction conditions: 10 mmol guaiacol, 5 mmol lauryl aldehyde, 100 mg P—SiO₂. Eₐ was 50 KJ mol⁻¹ for BAr and 72 kJ mol⁻¹ for the enal product. This difference in activation energies is due to different rate-determining steps in each reaction (References 38-39). Lower temperatures and longer reaction times promote selectivity.

TABLE 1
Reaction rates (M/min)

| | 130° C. | 140° C. | 150° C. |
|---|---|---|---|
| r_{BAR} | 0.0000381 | 0.0000453 | 0.0000784 |
| r_{Byproduct} | 0.00000676 | 0.0000109 | 0.0000189 |

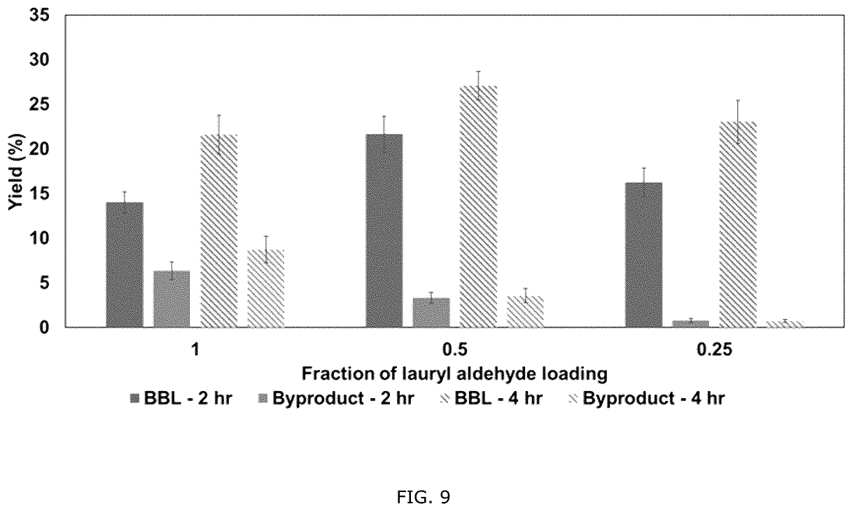

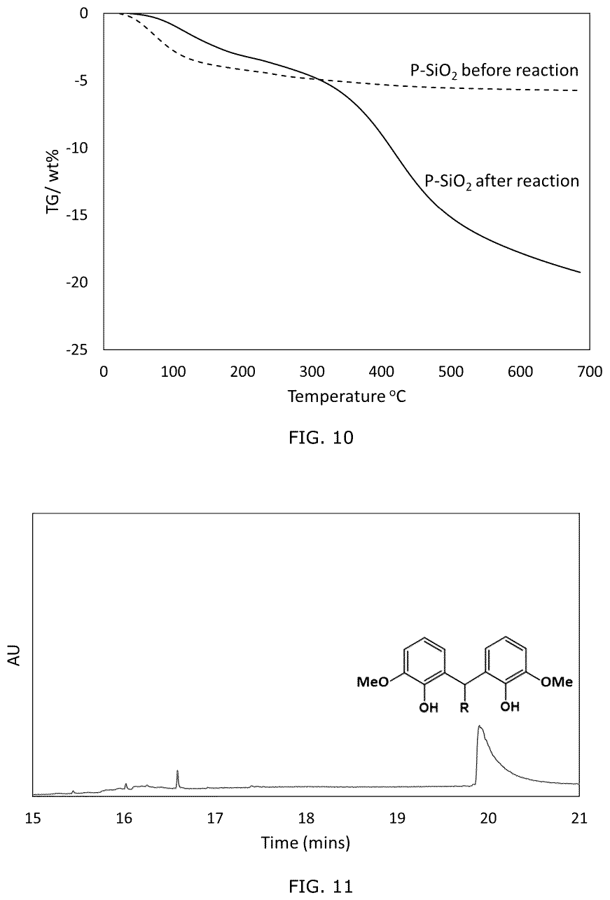

Optimal reaction conditions for the HAA reaction of guaiacol and lauryl aldehyde (150° C., 150 mg P—SiO₂, 12 hr) afforded yields of ˜40 wt. % BAR and ˜11 wt. % enal product, as summarized in Table 2 (Table S2). Hence, a semi-batch approach was adapted to minimize the enal product by periodically increasing the lauryl aldehyde in the reactor while providing the same total amount (see methods). As shown in FIG. 9, reducing the loading of lauryl aldehyde in each period lowers the yield of condensation byproduct from 8.7 to 0.7 wt. % at 4 hr and increases the BAR yield to 54 wt. % with a selectivity of 87% to the BAR product, a s summarized in Table 2. Increasing the reaction time or catalyst amount did not increase the guaiacol conversion beyond ˜60 wt. % and led to poor carbon balance (<65%). Without wishing to be bound by any particular theory, it is believed that this halt in guaiacol conversion can be attributed to P—SiO₂ deactivation as seen in recent literature (References5, 25, 39), which prevents further HAA reaction. TGA analysis (Heating from 30 to 700° C. at a heating rate of 10 K min⁻¹ under air (30 mL min⁻¹) of the spent catalyst showed significant mass loss compared to the fresh catalyst, attributed to oligomers and catalyst coking, as shown in FIG. 10. FIG. 11 shows GC analysis of the washed spent catalysts in THF also indicated BAR adsorbed onto the spent catalyst. Coke forms rapidly (within the first minutes), changing the catalyst color from clear to dark brown/black, as shown in FIG. 6. Thus, the present results support that a semi-batch operation would limit the concentration of aldehyde and minimize the condensation (side) reaction.

TABLE 2

| | Batch system | Semi-batch system |
|---|---|---|
| Guaiacol conversion (%) | 51.0 | 59.6 |
| Lauryl aldehyde conversion (%) | 96.2 | 94.0 |
| BAR yield (%) | 39.8 | 53.8 |
| Byproduct yield (%) | 11.0 | 6.8 |
| Carbon balance (%) | 85.0 | 92.0 |

### Catalyst Regeneration

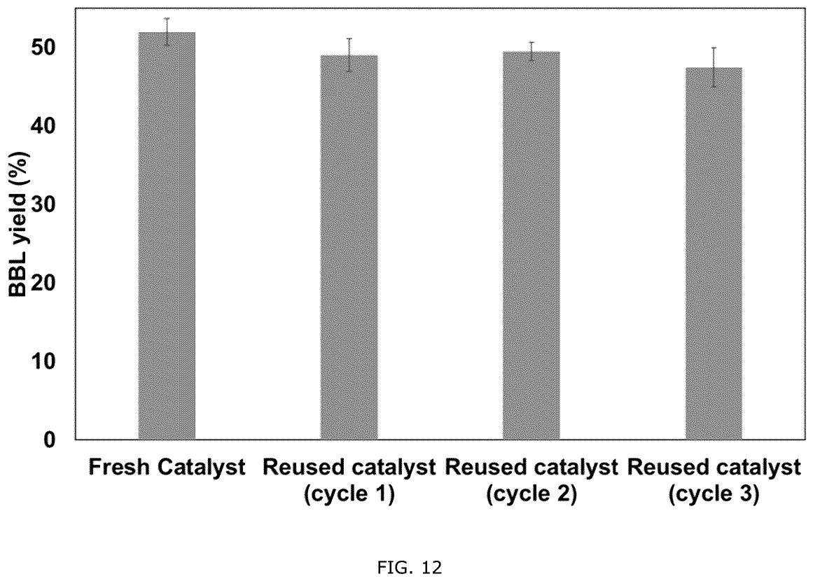

The spent catalyst was regenerated by (1) washing in cyclohexane thrice, next, (2) air-drying overnight, followed by (3) calcining of the washed and dried P—SiO₂ at 500° C. in air for 3 hr. The regenerated P—SiO₂ regained comparable performance to the fresh catalyst, as shown in FIG. 12. To overcome the plateau in yield stemming from the catalyst deactivation halting the HAA reaction, the reaction mixture was separated from the deactivated catalyst after 12 hr by centrifugation and decantation, was transferred to a reactor with fresh catalyst, and reacted with additional lauryl aldehyde (stoichiometric amount based on unreacted guaiacol), leading to ˜90 wt. % guaiacol conversion. The final HAA reaction product contained 76% BAr and 24% enal condensation product. These results support periodic or continuous catalyst regeneration in future work.

The HAA chemistry was exploited for several lignin-derived aromatic monomers, as summarized in Table 3 (Table 1) (Reaction conditions: 10 mmol substrate, 5 mmol lauryl aldehyde, 150° C., 12 hr, 150 mg P—SiO₂). It should be noted that phenols gave the highest conversion and selectivity to the BAr. Without wishing to be bound by any particular theory, it is believed that this could be due to their smaller steric hindrance in the mesoporous P—SiO₂ catalyst than guaiacols and syringols. A small alkyl group (methyl, ethyl) in the phenol increased the conversion, likely due to the electron-donation of these groups activating the ring. Longer alkyl side chains (propyl) on the phenol negatively affect reactivity, probably due to steric effects (Reference 5, 25). The presence of alkyl groups in guaiacols and syringols resulted in lower conversion and BAr selectivity. Methoxy and alkyl groups might sterically hinder the substrate from the catalyst sites and the lauryl aldehyde.

TABLE 3

| | | Conversion | Laurylaldehyde | BBL Yield (Selectivity)* |
|---|---|---|---|---|
| Substrate | | (%) | conversion (%) | (%) |
| Phenol | 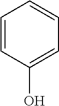 | 67 | 99.8 | 63 (91) |
| Methyl phenol | 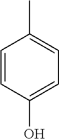 | 70 | 99.8 | 68 (93) |
| Ethyl phenol | 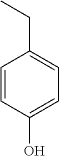 | 72 | 99.8 | 68 (95) |
| Propyl phenol | 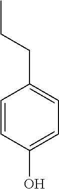 | 65 | 93.3 | 61 (86) |
| Guaiacol | 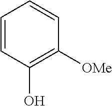 | 51 | 96.2 | 40 (78) |
| Methy lguaiacol | 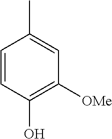 | 38 | 91.4 | 31 (64) |
| Ethyl guaiacol | 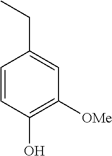 | 33 | 83.2 | 23 (43) |
| Propyl guaiacol | 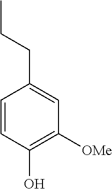 | 20 | 55.4 | 14 (38) |
| Syringol | 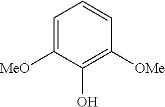 [SMILES: COc1cccc(OC)c1O] | 52 | 87.4 | 34 (67) |
| Methyl syringol | 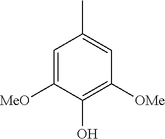 | 11 | 50.2 | 0 (0) |

*Reaction products: BBL and enal byproduct. Carbon balance > 85 wt. % (HA intermediate is added in the balance).

### HDO to Cycloaliphatic and Branched-Alkane Base Oils

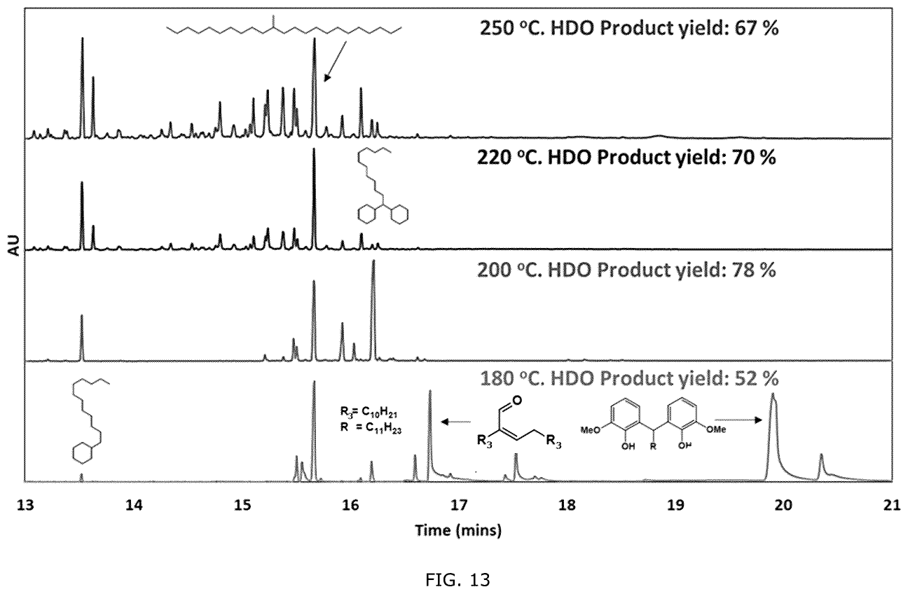

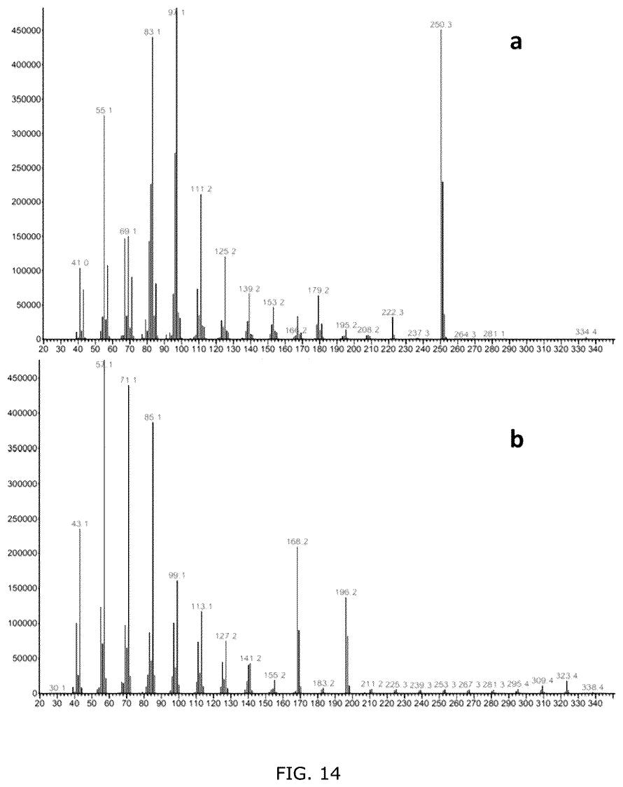

The BAR and enal HAA reaction product was dissolved in cyclohexane and transferred to a Parr reactor for HDO. Conditions employed in the production of jet fuels (Reference 40) and lubricants (Reference 5-8) were used and further optimized. Ir—ReOₓ/SiO₂ was selected due to its excellent HDO performance reported in our previous works (References 5-8, 40). At 180° C. used in prior work (References 5, 8), there remains unconverted BAr and enal byproduct, as shown in FIG. 13 (Reaction Conditions: 0.3 g HAA reaction product, 0.2 g Ir—ReOₓ on silica, 20 ml cyclohexane, 500 rpm, 18 hr, 5 MPa H₂), suggesting higher reaction temperature and/or longer reaction times for complete HDO. At 200° C., the BAr and enal byproduct were fully converted into cyclic C₂₄ alkane and branched aliphatic C₂₄ lubricant products, respectively, given the complete disappearance of the BAr (peaks at ˜20-20.5 mins) and enal byproduct (peaks at ˜17.6 mins), respectively, as shown in FIG. 13. Thus, FIG. 13 shows that at a reaction temperature if less than 200° C., there is insufficient hydrodeoxygenation and at a temperature equal to or greater than 200° C., there is more C—C cracking. ¹H NMR spectra after HDO, as shown in FIG. 14 corroborate the complete disappearance of aromatic signals (chemical shift 6.5-6.8 ppm) and consistency with the GC-MS data, shown in FIG. 15. At ≥220° C., BAR and enal byproduct are completely converted but also more C—C cracking products to dodecyl cyclohexane (peak at ˜13.5 mins) and shorter chain branched lubricants (side peaks between ˜15-15.5 mins), respectively. 200° C. was used for subsequent HDO experiments to minimize cracking while ensuring complete conversion.

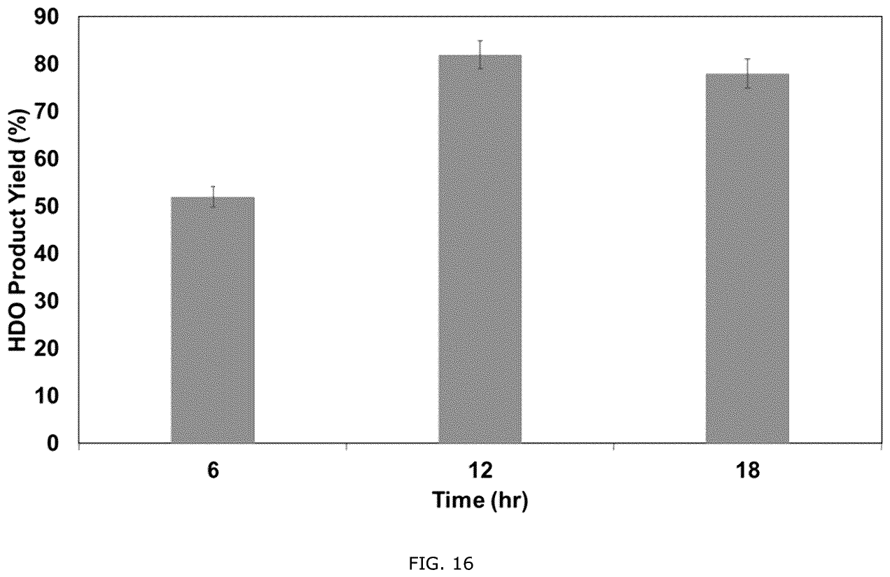

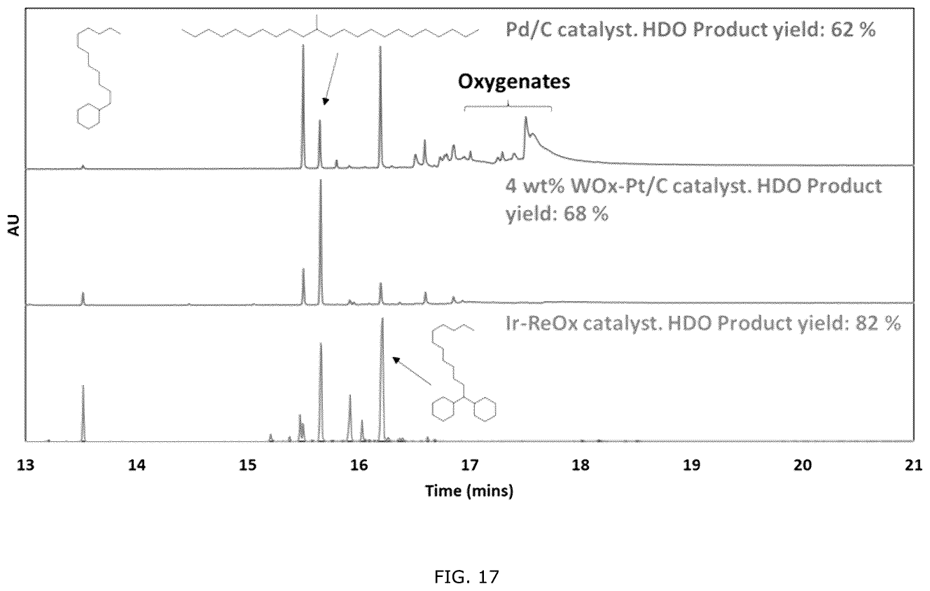

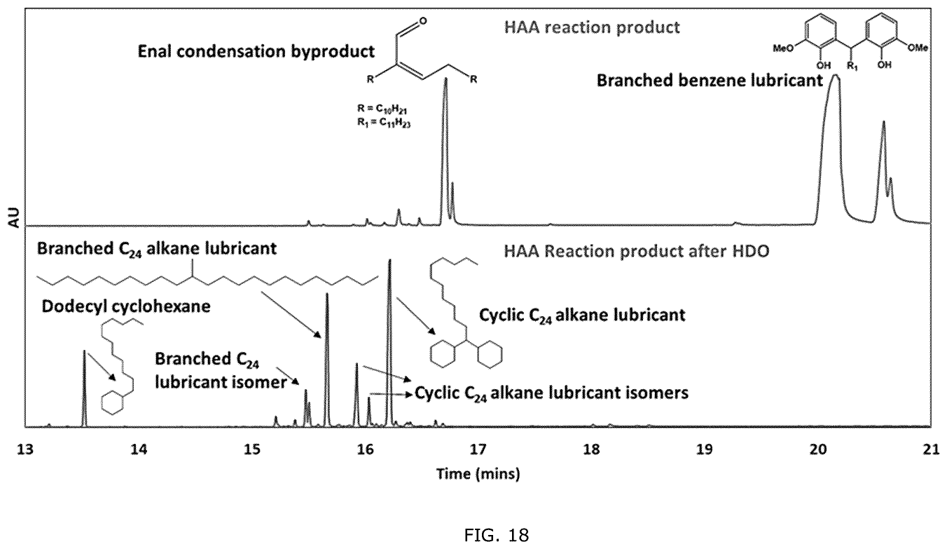

At 200° C., a reaction time of 6 hr was insufficient for complete HDO; 12 hr gave the highest lubricant yield (82%). Longer reaction times led to more C—C cracking and lower lubricant yields, as shown in FIG. 16. Other HDO catalysts (Pd/C and 4 wt. % WOx-Pt/C) afforded lower HDO lubricant yields, as shown in FIG. 17. The optimized reaction conditions afforded a lubricant mixture of 82% cyclic C₂₄ alkane and branched aliphatic C₂₄ base oil, with a distinct chromatogram, as shown in FIG. 18 from the HAA reaction product. GC analysis of the gas phase after HDO identified light alkanes (C₂H₆, C₃H₈, C₄H₈) from C—C cracking reactions. Small fractions of dodecyl cyclohexane and small alkanes from C-C cracking are also observed in the liquid phase. Notably, the biobased lubricant base oils according to embodiments of the present invention form selectively, minimizing expensive and complex separations associated with commercial mineral and synthetic base oils. A summary of all HDO results is summarized in Table 4 (Reaction conditions: 0.3 g HAA reaction product, 0.2 g catalyst, 20 ml cyclohexane, 500 rpm, 5 MPa H₂).

TABLE 4

| Time | Temperature | | C₂₄ lubricant mix yield* |
|---|---|---|---|
| (hr) | (° C.) | Catalyst | (%) |
| 6 | 200 | Ir—ReOx | 54 |
| 12 | 200 | Ir—ReOx | 82 |
| 18 | 200 | Ir—ReOx | 78 |
| 18 | 180 | Ir—ReOx | 52 |
| 18 | 220 | Ir—ReOx | 70 |
| 18 | 250 | Ir—ReOx | 67 |
| 12 | 200 | Pd/C | 62 |
| 12 | 200 | 4 wt. % WOx—Pt/C | 68 |

*contains C₂₄ cyclic alkane lubricant, C₂₄ branched alkane lubricant and dodecyl cyclohexane

Under similar reaction conditions, Pd/C still left oxygenated products. WOx-Pt/C cracked the branched cyclic lubricant product, reducing the C₂₄ alkane lubricant yield, likely due to the strong acidity of the catalyst. The excellent performance of the Ir—ReOx catalyst for HDO arises from a synergy between Ir and ReOx sites, whereby the Ir sites hydrogenate the benzene rings, and the acidic sites of ReOx activate the C—O bonds of the methoxy and phenolic groups. Selective HDO of lignin derivatives (phenols, cresols, and guaiacols) over metal/metal oxide catalysts has been demonstrated in recent works (References 29, 41, 42). It is believed that HDO and ring hydrogenation over these catalysts has not been reported before. Further simultaneous optimization of operating conditions and catalysts could further increase the yield for practical implementation.

### Lubricant Properties

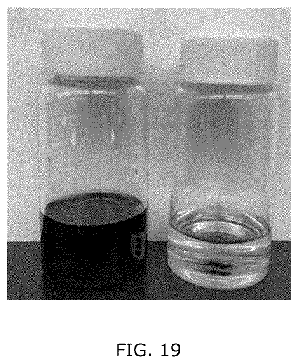

The HAA condensation product comprising BAr and enal compounds according to embodiments of the present invention is a highly viscous and dark-amber colored liquid at room temperature. After HDO, the obtained lubricant oil is less viscous and clear, as shown in FIG. 19 (HAA reaction conditions: 10 mmol guaiacol, 5 mmol lauryl aldehyde, 150° C., 12 h, 150 mg P—SiO₂; and HDO reaction conditions: 0.3 g HAA reaction product, 0.2 g Ir—ReOx/SiO₂ catalyst, 20 ml cyclohexane, 500 rpm, 200° C., 12 hr, 5 MPa H₂)

Important properties (kinematic viscosity, VI, and Noack volatility) of the base oils are compared with commercial mineral and alkylbenzene base oils, categorized by the American Petroleum Institute, are summarized in Table 5. Lubricant base oils should have high viscosities at high temperatures to create a thick hydrodynamic film between surfaces. At lower temperatures, base oils should be less resistant to flow, promoting fluidity. The viscosity index (VI), calculated using the KV₁₀₀ and KV₄₀ values, indicates the change in viscosity with temperature. A high viscosity index (VI) ensures lower dependence of lubricant viscosity on temperature, which is desirable for the lubricating film operating over a wide temperature range. Table 5 shows that the KV₁₀₀ and KV₄₀ of the base oils according to embodiments of the present invention are comparable to commercial Group III, IV and refrigerant base oils. The VI of our lubricant mix of cyclic and branched C₂₄ alkane base oil is above 100, indicating good lubricant quality.

TABLE 5
Properties of HAA reaction product and lubricant
mix of cyclic and branched C₂₄ alkane base oil compared
with those of select commercial lubricants.

| | | | | Noack |
|---|---|---|---|---|
| | KV₁₀₀ | KV₄₀ | | Volatility |
| Base oil | (cSt) | (cSt) | VI | (wt. %) |
| C₂₄ lubricant product mix | 3.18 | 12.6 | 118 | 37.10 |
| ExxonMobil PAO4 | 4.1 | 19.0 | 126 | 18.80 |
| Exxon Mobil SpectraSyn Plus | 3.6 | 15.4 | 120 | <17.0 |
| PAO-3.6 | | | | |
| HAA reaction product | 12.2 | 238 | −31 | 16.30 |
| Mobil EAL Arctic 220 | 18.1 | 220 | 88 | — |

The properties of commercial products were obtained from the product specifications datasheet disclosed by the manufacturers.

HAA reaction product contained 76% BAr and 24% enal product as shown below:

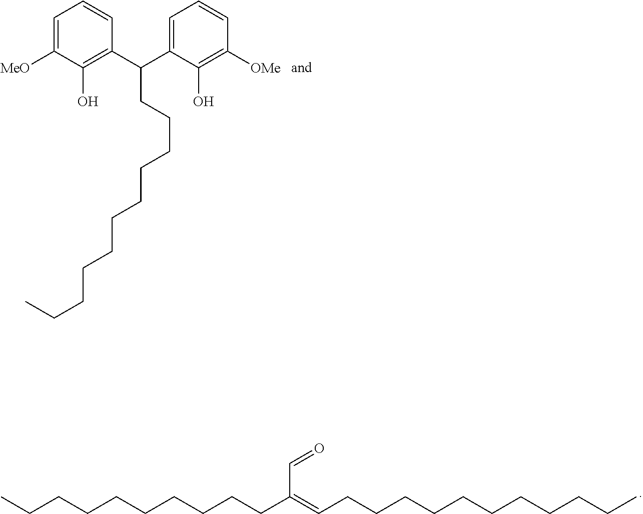 [SMILES: CCCCCCCCCCCC(c1cccc(OC)c1O)c1cccc(OC)c1O ; CCCCCCCCCCC/C=C(\C=O)CCCCCCCCCC]

The C₂₄ lubricant product mix contained 62% branched cyclic lubricant, 22% branched lubricant, 12% dodecyl cyclohexane, and 4% alkanes as shown below:

[symbol]

, ,and.

The high viscosity of the HAA reaction product according to embodiments of the present invention could be due to aromaticity, molecular structure, and oxygen in the phenolic and methoxy groups. These hydrophilic groups improve the polarity of base oils and enhance their solubility with polar additives for refrigerant applications (Reference 3). At higher temperatures, the viscosity of the HAA reaction product drops significantly, resulting in a negative viscosity index. This implies that the HAA reaction product rapidly thickens with decreasing temperature and thins out with increasing temperature. The high Noack volatility of the C₂₄ lubricant mix is attributed to the lighter hydrocarbon fragments in the base oil mix that evaporate under testing conditions. Conversely, the HAA reaction product has better volatility compared to commercial lubricants as it consists of high boiling point components.

According to embodiments of the present inventions, a new route to branched benzene lubricant (BAR) and branched cyclic lubricant (BCL) base oils from lignin-derived monomer guaiacol and fatty acid-derived aldehydes, such as lauryl aldehyde, using hydroxyalkylation/alkylation (HAA) followed by hydrodeoxygenation (HDO) is disclosed herein. P—SiO₂ was the best heterogeneous catalyst among several catalysts screened. HAA chemistry on the aromatic rings was found to be slower than on furans, requiring higher temperatures and enhancing side reactions. The main products of the HAA step were BAr and an aldol condensation enal product with a carbon balance of >85 wt. %. A semi-batch operation was introduced to minimize the enal product by limiting the amount of lauryl aldehyde, affording ˜90 wt. % guaiacol conversion and final HAA reaction product composition of 76% BAr and 24% enal condensation product at optimal reaction conditions (150° C., 150 mg P—SiO₂, 12 hr). According to embodiments of the present invention, this strategy can be extended to other lignin-related substrates, with activity and selectivity varying depending on functional groups and size. Subsequent HDO over an Ir—ReOₓ/SiO₂ catalyst (Re/Ir molar ratio=2) yielded 82% lubricant-ranged C₂₄ cyclic and branched alkanes base oils. A small fraction of low-carbon alkanes (>C₉) was also detected. Viscosity measurements indicates viscous properties comparable to petroleum-derived Group III, IV, and refrigerant base oils. Hence, the strategy to synthesize renewable base oils, as disclosed hereinabove could be used to replace petroleum-derived base oils and reduce the carbon footprint.

### REFERENCES

1 G. V. Research, Lubricants Market Size, Share| Industry Report, 2021-2028, https://www.grandviewresearch.com/industry-analysis/lubricants-market, (accessed 13 Apr. 2021).
2 C. P. Newswire, The global lubricants market size is projected to reach USD 182.6 billion by 2025 from USD 157.6 billion in 2020, at a CAGR of 3.0%, https://www.prnewswire.com/news-releases/the-global-lubricants-market-size-is-projected-to-reach-usd-182-6-billion-by-2025-from-usd-157-6-billion-in-2020 --at-a-cagr-of-3-0-301096072.html, (accessed 13 Apr. 2021).
3 K. Takigawa, S. I. Sandler and A. Yokozeki, Int. J. Refrig., 2002, 25, 1014-1024.
4 W. D. Cooper, R. C. Downing and J. B. Gray, Alkyl Benzene as a Compressor Lubricant, Wilmington, 1974.
5 S. Liu, T. R. Josephson, A. Athaley, Q. P. Chen, A. Norton, M. Ierapetritou, J. I. Siepmann, B. Saha and D. G. Vlachos, Sci. Adv., 2019, 5, 1-8.
6 S. Liu, B. Saha and D. G. Vlachos, Green Chem., 2019, 21, 3606-3614.
7 S. Liu, R. Bhattacharjee, S. Li, A. Danielson, T. Mazal, B. Saha and D. G. Vlachos, Green Chem., 2020, 22, 7896-7906.
8 A. M. Norton, S. Liu, B. Saha and D. G. Vlachos, ChemSusChem, 2019, 12, 4780-4785.
9 S. Van Den Bosch, W. Schutyser, R. Vanholme, T. Driessen, S. F. Koelewijn, T. Renders, B. De Meester, W. J. J. Huijgen, W. Dehaen, C. M. Courtin, B. Lagrain, W. Boerjan and B. F. Sels, Energy Environ. Sci., 2015, 8, 1748-1763.
10 O. E. Ebikade, N. Samulewicz, S. Xuan, J. D. Sheehan, C. Wu and D. G. Vlachos, Green Chem., 2020, 22, 7435-7447.
11 Y. M. Questell-Santiago, M. V Galkin, K. Barta and J. S. Luterbacher, Nat. Rev. Chem., DOI: 10.1038/s41570-020-0187-y.
12 R. M. O'dea, J. A. Willie and T. H. Epps, ACS Macro Lett., 2020, 9, 476-493.
13 S. Elangovan, A. Afanasenko, J. Haupenthal, Z. Sun, Y. Liu, A. K. H. Hirsch and K. Barta, ACS Cent. Sci., 2019, 5, 1707-1716.
14 J. Gierer, Wood Sci. Technol., 1985, 19, 289-312.
15 D. S. Bajwa, G. Pourhashem, A. H. Ullah and S. G. Bajwa, Ind. Crops Prod., 2019, 139, 111526.
16 L. Dessbesell, M. Paleologou, M. Leitch, R. Pulkki and C. (Charles) Xu, Renew. Sustain. Energy Rev., 2020, 123, 109768.
17 O. Ajao, J. Jeaidi, M. Benali, A. Restrepo, N. El Mehdi and Y. Boumghar, Molecules, 2018, 23, 377.
18 O. Ajao, J. Jeaidi, M. Benali, O. Y. Abdelaziz and C. P. Hulteberg, Bioresour. Technol., DOI: 10.1016/j.biortech.2019.121799.
19 Chem. Eng. News, 1957, 35, 28-30.
20 C. L. Chambon, M. Chen, P. S. Fennell and J. P. Hallett, Front. Chem., 2019, 7, 246.
21 L. Shuai, M. T. Amiri, Y. M. Questell-Santiago, F. Héroguel, Y. Li, H. Kim, R. Meilan, C. Chapple, J. Ralph and J. S. Luterbacher, Science, 2016, 354, 329-334.
22 E. M. Anderson, M. L. Stone, R. Katahira, M. Reed, G. T. Beckham and Y. Román-Leshkov, Joule, 2017, 1, 613-622.
23 S. Wang, L. Shuai, B. Saha, D. G. Vlachos and T. H. Epps, ACS Cent. Sci., 2018, 4, 701-708.
24 Y. Liao, S. F. Koelewijn, G. van den Bossche, J. van Aelst, S. van den Bosch, T. Renders, K. Navare, T. Nicolaï, K. van Aelst, M. Maesen, H. Matsushima, J. M. Thevelein, K. van Acker, B. Lagrain, D. Verboekend and B. F. Sels, Science (80-.)., 2020, 367, 1385-1390.
25 P. Ferrini, S. F. Koelewijn, J. Van Aelst, N. Nuttens and B. F. Sels, ChemSusChem, 2017, 10, 2249-2257.
26 S. Sadula, N. Rodriguez Quiroz, A. Athaley, O. E. Ebikade, M. Ierapetritou, D. Vlachos and B. Saha, Green Chem., 2021, 23, 1200-1211.
27 T. Yokoyama and N. Yamagata, Appl. Catal. A Gen., 2001, 221, 227-239.
28 C. Wang, J. D. Lee, Y. Ji, T. M. Onn, J. Luo, C. B. Murray and R. J. Gorte, Catal. Letters, 2018, 148, 1047-1054.
29 C. Wang, A. V. Mironenko, A. Raizada, T. Chen, X. Mao, A. Padmanabhan, D. G. Vlachos, R. J. Gorte and J. M. Vohs, ACS Catal., 2018, 8, 7749-7759.
30 K. E. Horner and P. B. Karadakov, J. Org. Chem., 2013, 78, 8037-8043.
31 P. Kumar, M. Varkolu, S. Mailaram, A. Kunamalla and S. K. Maity, in Polygeneration with Polystorage: For Chemical and Energy Hubs, Elsevier, 2018, pp. 373-407.
32 N. Barthel, A. Finiels, C. Moreau, R. Jacquot and M. Spagnol, J. Mol. Catal. A Chem., 2001, 169, 163-169.
33 G. M. Loudon, in Organic Chemistry, 5th edn., 2009, pp. 1-1374.
34 H. A. Meylemans, T. J. Groshens and B. G. Harvey, ChemSusChem, 2012, 5, 206-210.
35 S. Zhao and M. M. Abu-Omar, ACS Sustain. Chem. Eng., 2016, 4, 6082-6089.
36 M. Balakrishnan, G. E. Arab, O. B. Kunbargi, A. A. Gokhale, A. M. Grippo, F. D. Toste and A. T. Bell, Green Chem., 2016, 18, 3577-3581.
37 H. J. Cho, M. J. Kuo, M. Ye, Y. Kurz, Y. Yuan and R. F. Lobo, ACS Sustain. Chem. Eng., 2021, acssuschemeng.0c09227.
38 G. Li, N. Li, Z. Wang, C. Li, A. Wang, X. Wang, Y. Cong and T. Zhang, ChemSusChem, 2012, 5, 1958-1966.
39 A. N. Migues, S. Vaitheeswaran and S. M. Auerbach, J. Phys. Chem. C, 2014, 118, 20283-20290.
40 S. Liu, T. Simonetti, W. Zheng and B. Saha, ChemSusChem, 2018, 11, 1446-1454.
41 S. Koso, I. Furikado, A. Shimao, T. Miyazawa, K. Kunimori and K. Tomishige, Chem. Commun., 2009, 2035-2037.
42 Y. Nakagawa and K. Tomishige, Catal. Today, 2012, 195, 136-143.
43 B. Xiao, M. Zheng, X. Li, J. Pang, R. Sun, H. Wang, X. Pang, A. Wang, X. Wang and T. Zhang, Green Chem., 2016, 18, 2175-2184.
44 J. He, S. P. Burt, M. Ball, D. Zhao, I. Hermans, J. A. Dumesic and G. W. Huber, ACS Catal., 2018, 8, 1427-1439.
45 E. Soghrati, C. K. Poh, Y. Du, F. Gao, S. Kawi and A. Borgna, ChemCatChem, 2018, 10, 4652-4664.
46 K. J. Stephens, A. M. Allgeier, A. L. Bell, T. R. Carlson, Y. Cheng, J. T. Douglas, L. A. Howe, C. A. Menning, S. A. Neuenswander, S. K. Sengupta, P. S. Thapa and J. C. Ritter, ACS Catal., 2020, 10, 12996-13007.
47 R. Shu, R. Li, B. Lin, C. Wang, Z. Cheng and Y. Chen, Biomass and Bioenergy, 2020, 132, 105432.
48 Y. Jing and Y. Wang, Front. Chem. Eng., 2020, 2, 10.
49 T. Yokoyama, N. Yamagata, Appl. Catal. A: Gen. 221, 227-239 (2001).
50 Y. Nakagawa, M. Tamura, K. Tomishige, ACS Catal. 3, 2655-2668 (2013).

What is claimed:

1. A bio-based composition comprising a branched aromatic compound having one of the following structures: 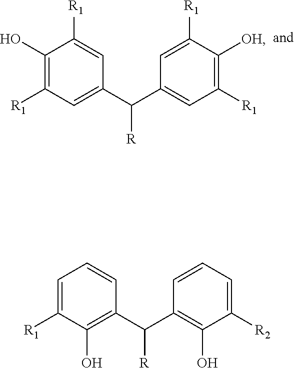
wherein R is an alkyl group having 1 to 20 carbon atoms, wherein the alkyl group is a linear, a branched, or a cycloalkyl group, and wherein R₁ and R₂ are independently selected from hydrogen and a methoxy group.

2. The bio-based composition of claim 1, further comprising a bio-based aliphatic enal compound having the following structure: 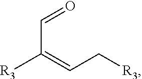
wherein each R₃ is independently selected from an alkyl group having 0 to 19 carbon atoms, and wherein the alkyl group is a linear, a branched, or a cycloalkyl group.

3. A bio-based composition comprising a branched cycloaliphatic compound having one of the following structures: 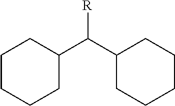
and isomers thereof, and 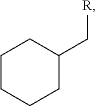
wherein R is an alkyl group having 1 to 20 carbon atoms, and wherein the alkyl group is a linear, a branched, or a cycloalkyl group.

4. The bio-based composition of claim 3, further comprising a branched aliphatic compound having the following formula: 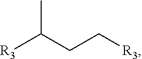
and isomers thereof,
wherein each R₃ is independently selected from an alkyl group having 0 to 19 carbon atoms, and wherein the alkyl group is a linear, a branched, or a cycloalkyl group.

5. The composition according to claim 1, wherein at least one of the compounds has a bio-based content in the range of 20 to 100%, according to ASTM-D6866.

6. A bio-based lubricant composition comprising:
(i) a branched aromatic compound having one of the following structures: 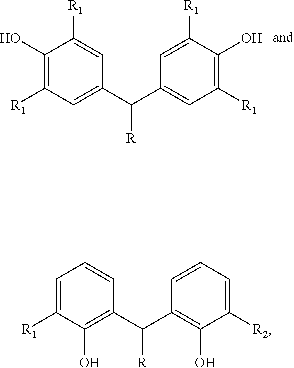
and optionally an aliphatic enal compound having the following structure: 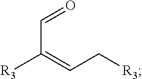
or
(ii) a branched cycloaliphatic compound having one of the following structures: 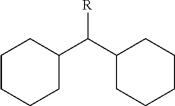
and isomers thereof, and 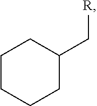
and optionally a branched aliphatic compound having the following structure: 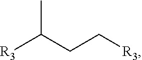
and isomers thereof,
wherein R is an alkyl group having 1 to 20 carbon atoms, wherein the alkyl group is a linear, a branched, or a cycloalkyl group, wherein R₁ and R₂ are independently selected from hydrogen and methoxy group, and wherein each R₃ is an alkyl group having 0 to 19 carbon atoms; and
(ii) an effective amount of one or more additives.

7. The bio-based lubricant composition according to claim 6, wherein the one or more additives are selected from the group consisting of antioxidants, stabilizers, detergents, dispersants, demulsifiers, antioxidants, anti-wear additives, pour point depressants, viscosity index modifiers, friction modifiers, anti-foam additives, defoaming agents, corrosion inhibitors, wetting agents, rust inhibitors, copper passivators, metal deactivators, extreme pressure additives, and combinations thereof.

8. The bio-based lubricant composition according to claim 6, further comprising one or more co-base oils selected from the group consisting of API Group I base oil, Group II base oil, Group III base oil, Group IV base oil, Group V base oil, gas-to-liquid (GTL) base oil, and combinations thereof.

9. The bio-based lubricant composition according to claim 6, wherein the lubricant composition has a kinematic viscosity at 100° C. in the range of 2 to 100 CSt, as measured by ASTM D445.

10. The bio-based lubricant composition according to claim 6, wherein the lubricant composition has a kinematic viscosity at 40° C. in the range of 5 to 300 CSt, as measured by ASTM D445.

11. The bio-based lubricant composition according to claim 6, wherein the lubricant composition has a viscosity index calculated from kinetic viscosity at 100° C. and 40° C., in the range of −40 to 200, as measured by ASTM D2270.

12. (canceled)

13. A method comprising carrying out hydroxyalkylation/alkylation of a lignin-derived monomer containing a phenolic hydroxyl group and an aldehyde (RCHO) in the presence of a hyrdoxyalkylation/alkylation acidic catalyst to form a bio-based branched aromatic compound having one of the following structures: 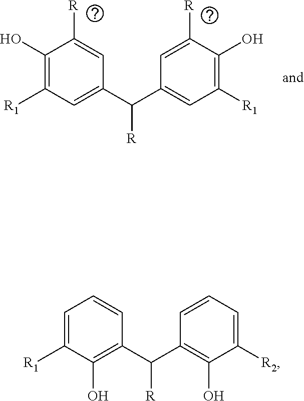
and optionally a branched aliphatic enal compound having the following structure: 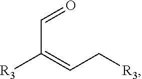
wherein R is an alkyl group having 1 to 20 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19 carbon atoms.

14. The method according to claim 13, wherein the lignin-derived monomer comprises:
(i) an unsubstituted phenol or a substituted phenol, such as cresol (methyl phenol), ethylphenol, propylphenol,
(ii) a monomethoxyphenol, such as guaiacol (monomethoxy-substituted phenol), methylguaiacol, ethylguaiacol, propylguaiacol,
(iii) a dimethoxyphenol, such as a syringol (dimethoxy-substituted phenol), methyl syringol, or
(iv) combinations thereof.

15. The method according to claim 13 further comprising hydrodeoxygenating the bio-based aromatic compound in the presence of a hydrodeoxygenation catalyst to obtain a bio-based cycloaliphatic compound having the following formula: 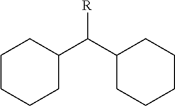
wherein R is an alkyl group having 1 to 20, or 3 to 19, or 5 to 17 carbon atoms, and wherein the alkyl group is a linear, a branched, or a cycloalkyl group.

16. The method according to claim 13, wherein the hydroxyalkylation/alkylation acidic catalyst is a homogeneous catalyst comprising sulfuric acid, methanesulfonic acid, acetic acid, triflic acid, or p-toluenesulfonic acid.

17. The method according to claim 13, wherein the hydroxyalkylation/alkylation acidic catalyst is a solid acid catalyst comprising perfluorinated sulfonic acid resins, sulfonic acid-functionalized cross-lined polystyrene resins, zeolites, or silica supported H₃PO₄.

18. The method according to claim 15, wherein the hydrodeoxygenation catalyst is a solid acid supported metal-based catalyst selected from Ni/ZSM-5, Pd/ZSM-5, Pd/BEA, or a physical mixture of a metal based catalyst with a solid acid, including Pd/C+ZSM-5, Pd/C+BEA, Pt/C+BEA, Pt-WOx/C and preferably a supported metal-metal oxide catalyst.

19. The method according to claim 18, wherein the supported metal-metal oxide catalyst comprises Ir—ReOₓ/SiO₂, Ir—MoOₓ/SiO₂ or ¹M²MO/SiO₂, wherein ¹M=Ir, Ru, Ni, Co, Pd, Pt, or Rh and ²M=Re, Mo, W, Nb, Mn, V, Ce, Cr, Zn, Co, Y, or Al.

20. (canceled)

21. A personal care composition comprising:
a) a base oil comprising one or more of the following:
(i) a branched aromatic compound having one of the following structures: 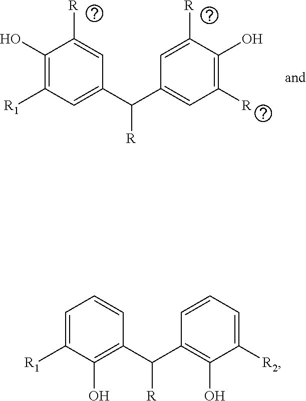
and optionally an aliphatic enal compound having the following structure: 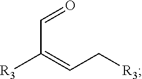
or
(ii) a branched cycloaliphatic compound having one of the following structures: 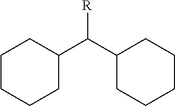
and isomers thereof, and 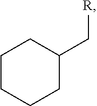
and optionally a branched aliphatic compound having the following structure: 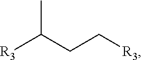
and isomers thereof,
wherein R is an alkyl group having 1 to 20 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19 carbon atoms;
b) an effective amount of one or more additives selected from the group consisting of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, and water; and
c) optionally, an active ingredient selected from the group consisting of antibiotic, antiseptic, antifungal, corticosteroid, and anti-acne agent.

22. A pharmaceutical composition comprising:
(a) a base oil comprising one or more of the following:
(i) a branched aromatic compound having one of the following structures: 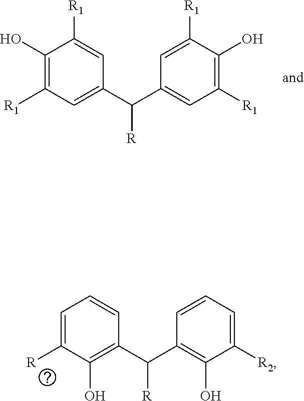
and optionally an aliphatic enal compound having the following structure: 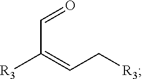
or
(ii) a branched cycloaliphatic compound having one of the following structures: 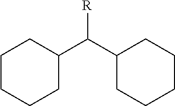
and isomers thereof, and 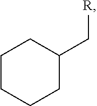
and optionally a branched aliphatic compound having the following structure: 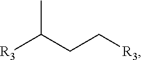
and isomers thereof,
wherein R is an alkyl group having 1 to 20 carbon atoms, wherein the alkyl group is a linear, a branched, or a cycloalkyl group, wherein R₁ and R₂ are independently selected from hydrogen and a methoxy group, and wherein each R₃ is an alkyl group having 0 to 19 carbon atoms;
(b) an effective amount of one or more pharmaceutically active ingredients; and
(c) optionally, one or more pharmaceutically acceptable excipients.

23. The composition according to claim 3, wherein at least one of the compounds has a bio-based content in the range of 20 to 100%, according to ASTM-D6866.
